(19) United States
(12) Patent Application Publication
(10) Pub. No.: US 2022/0125868 A1
(43) Pub. Date: Apr. 28, 2022

(54) HEMP COMPOSITIONS AND METHODS OF MAKING THE SAME

(71) Applicant: William Landers, Captain Cook, HI (US)

(72) Inventor: William Landers, Captain Cook, HI (US)

(21) Appl. No.: 17/452,029

(22) Filed: Oct. 22, 2021

Related U.S. Application Data
(60) Provisional application No. 63/104,232, filed on Oct. 22, 2020.

(51) Int. Cl. A61K 36/185; A23L 33/00; A23L 33/105; A23L 3/54; A23L 2/52; A23F 5/02; A61K 9/00; A61K 36/74; A61K 36/752

(52) U.S. Cl. CPC A61K 36/185; A23L 33/40; A23L 33/105; A23L 3/54; A23V 2002/00; A23F 5/02; A61K 9/0095; A61K 36/74; A61K 36/752; A23L 2/52

(57) ABSTRACT

The present disclosure provides new beverages and methods of making said beverages from hemp juice, coffee fruit, and oranges. The present disclosure also provides new beverages and methods of making said beverages from hemp juice and coffee fruit juice. The disclosure further relates to the production of a non-water-soluble and water-soluble coffee fruit, hemp juice, and orange powders.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world. Further, U.S. Patent Application Publication US 2016/0030350 A1, published on Feb. 4, 2016, and entitled: Preparation of Coffee-based Extracts and Powders, is hereby incorporated by reference. The present application also claims the benefit of U.S. Provisional Application No. 63/104,232, filed on Oct. 22, 2020, and entitled: Hemp Compositions and Methods of Making the Same. Each of the foregoing applications is incorporated by reference in their entirety herein.

### FIELD

The present application relates to products combining juice or parts of coffee fruit and/or orange with Cannabis (hemp). The resulting products can be used as a food product or dietary supplement. The disclosure further relates to methods of producing such products. Dried orange pulp power, and methods of producing the same are also disclosed.

### BACKGROUND OF THE INVENTION

The fruit of the coffee plant (e.g., Coffea arabica), often called the “coffee cherry” or “coffee fruit,” contains a coffee bean, which is utilized to produce coffee. The remaining portion of the coffee fruit is traditionally viewed as waste, although the mesocarp, or “pulp” is sometimes processed into compost or animal food. The pulp is high in polyphenols, such as the chlorogenic acids caffeic acid, quinic acid, ferulic acid, proanthocyanidins, and others. The antioxidant activity of polyphenols slows or prevents damage to cells, or “oxidative stress” caused by free radicals. Oxidative stress is associated with several disease states including diabetes, Alzheimer's Disease, cancer, atherosclerosis, and vision loss.

The hemp plant is utilized in industrial fiber production. The leafy upper part of the hemp plant, including the flower heads, also called inflorescences, contain high concentrations of cannabidiol (CBD), terpenoids, and flavonoids. Terpenoids, such as myrcene, pinene, limonene, linalool, and eucalyptol, have anti-inflammatory, anti-microbial, and anti-tumoral effects; flavonoids have anti-inflammatory properties. Cannabinoids, such as cannabidiol, are associated with health benefits such as pain relief and reduction of anxiety, depression, migraines, and acne.

Orange pulp is often discarded industrially after orange juice production. Orange pulp is a rich source of nutrients, including antioxidants, vitamin A, vitamin C, folate, potassium, and fiber. The nutrients in orange pulp are associated with health benefits, such as a reduction m inflammation, improvements in circulation, and boosts in immunity and metabolism

The health benefits, which derive from the presence of the medically relevant compounds, within hemp, coffee fruit, and oranges have not been realized. As such, there remains a need in the art for the production of products, which contain hemp, coffee fruit, and oranges.

### SUMMARY OF THE INVENTION

Beverages and methods for making a beverage from coffee fruit and hemp are described herein.

In some aspects, a water infusing powder comprising: dehydrated coffee fruit pulp powder and dehydrated hemp pulp powder is described herein.

In some embodiments, the dehydrated coffee fruit pulp powder and dehydrated hemp pulp powder are dehydrated using an ultrasound pulverization system.

In some embodiments, the water infusing powder comprises dehydrated coffee fruit pulp powder and dehydrated hemp pulp powder, wherein the dehydrated pulp is from a hemp inflorescence.

In some embodiments, the water infusing powder comprises dehydrated coffee fruit pulp powder and dehydrated hemp pulp powder, wherein the dehydrated hemp pulp powder comprises dehydrated pulp from a hemp sun leaf In some embodiments, the water infusing powder comprises dehydrated coffee fruit pulp powder and dehydrated hemp pulp powder, wherein the dehydrated coffee fruit pulp powder is substantially free of dehydrated coffee bean powder.

In some embodiments, the water infusing powder comprises dehydrated coffee fruit pulp powder and dehydrated hemp pulp powder, wherein the dehydrated coffee fruit pulp powder comprises dehydrated coffee bean powder.

In some aspects, a method for producing a water infusing powder is described herein, said method comprising: providing hemp pulp, coffee fruit pulp, and ascorbic acid in a container to produce a mixture and dehydrating the mixture to create a water infusing dried powder.

In some aspects, a water-soluble powder comprising dehydrated coffee fruit juice and dehydrated hemp juice is described herein.

In some embodiments, the water-soluble powder comprises dehydrated coffee fruit juice and dehydrated hemp juice, wherein the dehydrated coffee fruit juice had a sugar content between 2° Bx to 8° Bx before being dehydrated.

In some embodiments, the water-soluble powder comprises dehydrated coffee fruit juice and dehydrated hemp juice, wherein the dehydrated coffee fruit juice had a sugar content between 3° Bx to 4° Bx before being dehydrated.

In some embodiments, the water-soluble powder comprises dehydrated coffee fruit juice and dehydrated hemp juice, wherein the dehydrated hemp juice comprises dehydrated juice from hemp inflorescences.

In some embodiments, the water-soluble powder comprises dehydrated coffee fruit juice and dehydrated hemp juice, wherein the dehydrated hemp juice comprises dehydrated juice from hemp sun leaves.

In some embodiments, the water-soluble powder comprises dehydrated coffee fruit juice and dehydrated hemp juice, wherein the dehydrated coffee fruit juice comprises dehydrated juice from whole coffee fruits.

In some embodiments, the water-soluble powder comprises dehydrated coffee fruit juice and dehydrated hemp juice, wherein the dehydrated coffee fruit juice comprises dehydrated juice from debeaned coffee fruits.

In some aspects, a method for producing a water-soluble powder is described, wherein said method comprises drying coffee fruit juice to produce dried coffee fruit powder, drying hemp juice to produce dried hemp juice powder, and combining the hemp juice powder with the dried coffeefruit powder to form a water-soluble dried powder.

In some aspects, a method for producing a water-soluble powder is described, wherein said method comprises drying premixed coffee fruit juice and hemp juice to form a water-soluble dried powder.

In some embodiments, the method for producing a water-soluble powder comprises coffee fruit juice that is fortified with coffee fruit extract.

In some aspects, a hemp and coffee fruit beverage is described, wherein the beverage comprises coffee fruit juice and hemp juice.

In some embodiments, a hemp and coffee fruit beverage is described, wherein the beverage comprises coffee fruit juice, hemp juice, and sugar.

In some embodiments, a hemp and coffee fruit beverage is described in which the coffee fruit juice and hemp juice are mixed in a ratio of 2:1 w/w (w/w meaning weight to weight, in this example 2 weights of coffee fruit to 1 weight of hemp juice) to 4:1 w/w.

In some embodiments, a hemp and coffee fruit beverage is described in which the coffee fruit juice and hemp juice are mixed in a ratio of 3:1 w/w.

In some embodiments, a hemp and coffee fruit beverage is described, wherein the beverage comprises coffee fruit juice and hemp juice, wherein the hemp juice comprises juice from hemp inflorescences.

In some embodiments, a hemp and coffee fruit beverage is described, wherein the beverage comprises coffee fruit juice and hemp juice, wherein the hemp juice comprises juice from hemp sun leaves.

In some aspects, a beverage is prepared by a method comprising contacting a water infusing powder with water.

In some embodiments, a beverage is prepared by a method comprising a water-soluble dried powder with water.

In some embodiments, the beverages described herein comprise lemon juice and ginger.

In some aspects, a water-permeable container comprising a water infusing powder is described herein.

In some aspects, a gel capsule comprising a water infusing powder is described herein.

In some aspects, a gel capsule comprising a water-soluble powder is described herein.

In some aspects, a tablet comprising a water infusing powder is described herein.

In some aspects, a tablet comprising a water-soluble powder is described herein.

In some aspects, a water infusing powder comprising: dehydrated coffee fruit pulp powder, dehydrated hemp pulp powder, and dehydrated orange pulp powder is described herein.

In some embodiments, the dehydrated coffee fruit pulp powder, dehydrated hemp pulp powder, and dehydrated orange pulp powder are dehydrated using an ultrasound pulverization system.

In some embodiments, the water infusing powder comprises dehydrated coffee fruit pulp powder, dehydrated hemp pulp powder, and dehydrated orange pulp powder, wherein the dehydrated hemp pulp is from a hemp inflorescence.

In some embodiments, the water infusing powder comprises dehydrated coffee fruit pulp powder, dehydrated hemp pulp powder, and dehydrated orange pulp powder, wherein the dehydrated hemp pulp is from a hemp sun leaf In some embodiments, the water infusing powder comprises dehydrated coffee fruit pulp powder, dehydrated hemp pulp powder, and dehydrated orange pulp powder, wherein the dehydrated coffee fruit pulp powder is substantially free of dehydrated coffee bean powder.

In some embodiments, the water infusing powder comprises dehydrated coffee fruit pulp powder, dehydrated hemp pulp powder, and dehydrated orange pulp powder, wherein the dehydrated coffee fruit pulp powder comprises dehydrated coffee bean powder.

In some embodiments, the water infusing powder comprises dehydrated coffee fruit pulp powder, dehydrated hemp pulp powder, and dehydrated orange pulp powder, wherein the dehydrated orange pulp powder comprises a whole orange.

In some embodiments, the water infusing powder comprises dehydrated coffee fruit pulp powder, dehydrated hemp pulp powder, and dehydrated orange pulp powder, wherein the dehydrated orange pulp powder comprises a peeled orange.

In some embodiments, the water infusing powder comprises dehydrated coffee fruit pulp powder, dehydrated hemp pulp powder, and dehydrated orange pulp powder, wherein the dehydrated orange pulp powder comprises a juiced orange.

In some embodiments, the water infusing powder comprises dehydrated coffee fruit pulp powder, dehydrated hemp pulp powder, and dehydrated orange pulp powder, wherein the dehydrated orange pulp powder comprises a juiced orange without the peel.

In some aspects, the disclosure describes a method for producing a water infusing powder, said method comprising providing hemp pulp, coffee fruit pulp, and orange pulp in a container to produce a mixture and dehydrating the mixture to create a water infusing dried powder.

In some embodiments, the disclosure describes a method for producing a water infusing powder, said method comprising providing hemp pulp, coffee fruit pulp, orange pulp, and ascorbic acid in a container to produce a mixture and dehydrating the mixture to create a water infusing dried powder.

In some aspects, a water-soluble powder comprising: dehydrated coffee fruit juice, dehydrated hemp juice, and dehydrated orange juice powder is described herein.

In some embodiments, a water-soluble powder comprising: dehydrated coffee fruit juice, dehydrated hemp juice, and dehydrated orange juice powder, wherein the dehydrated coffee fruitjuice has a sugar content between 2° Bx to 8° Bx before being dehydrated is described herein.

In some embodiments, a water-soluble powder comprising: dehydrated coffee fruit juice, dehydrated hemp juice, and dehydrated orange juice powder, wherein the dehydrated coffee fruitjuice has a sugar content between 3° Bx to 4° Bx before being dehydrated is described herein.

In some embodiments, a water-soluble powder comprising: dehydrated coffee fruit juice, dehydrated hemp juice, and dehydrated orange juice powder, wherein the dehydrated hemp juice comprises dehydrated juice from hemp inflorescences is described herein.

In some embodiments, a water-soluble powder comprising: dehydrated coffee fruit juice, dehydrated hemp juice, and dehydrated orange juice powder, wherein the dehydrated hemp juice comprises dehydrated juice from hemp sun leaves is described herein.

In some embodiments, a water-soluble powder comprising: dehydrated coffee fruit juice, dehydrated hemp juice, and dehydrated orange juice powder, wherein the dehydrated coffee fruitjuice comprises dehydrated juice from whole coffee fruits is described herein.

In some embodiments, a water-soluble powder comprising: dehydrated coffee fruit juice, dehydrated hemp juice, and dehydrated orange juice powder, wherein the dehydrated coffee fruitjuice comprises dehydrated juice from debeaned coffee fruits is described herein.

In some embodiments, a water-soluble powder comprising: dehydrated coffee fruit juice, dehydrated hemp juice, and dehydrated orange juice powder, wherein the dehydrated orange juice powder comprises dehydrated juice from a whole orange is described herein.

In some embodiments, a water-soluble powder comprising: dehydrated coffee fruit juice, dehydrated hemp juice, and dehydrated orange juice powder, wherein the dehydrated orange juice powder comprises dehydrated juice from a peeled orange is described herein.

In some aspects, a method for producing a water-soluble powder, said method comprising: drying coffee fruit juice to produce dried coffee fruit powder; drying hemp juice to produce dried hemp juice powder; drying orange juice to produce a dried orange juice powder; and combining the hemp juice powder, dried coffee fruit powder, and dried orange juice powder to form a water-soluble dried powder, is described herein.

In some aspects, a method for producing a water-soluble powder, said method comprising: drying premixed coffee fruit powder, hemp juice, and orange juice to form a water-soluble dried powder, is described herein.

In some embodiments, a method for producing a water-soluble powder, said method comprising: drying coffee fruit juice to produce dried coffee fruit powder; drying hemp juice to produce dried hemp juice powder; drying orange juice to produce a dried orange juice powder; and combining the hemp juice powder, dried coffee fruit powder, and dried orange juice powder to form a water-soluble dried powder, wherein the coffee fruit juice is fortified with coffee fruit extract as described herein.

In some embodiments, a method for producing a water-soluble powder, said method comprising: drying premixed coffee fruit juice powder, hemp juice, and orange juice to form a water-soluble dried powder, wherein the coffee fruit is fortified with coffee fruit extract is described herein.

In some aspects, a hemp, coffee fruit, and orange beverage comprising coffee fruit juice, hemp juice, and orange juice is described herein.

In some embodiments, a hemp, coffee fruit, and orange beverage comprising coffee fruit juice, hemp juice, and orange juice further comprising sugar is described herein.

In some embodiments, a hemp, coffee fruit, and orange beverage comprising coffee fruit juice, hemp juice, and orange juice, wherein the coffee fruit juice and hemp juice are mixed in a ratio of 2:1 w/w to 4:1 w/w, is described herein.

In some embodiments, a hemp, coffee fruit, and orange beverage comprising coffee fruit juice, hemp juice, and orange juice, wherein the coffee fruit juice and hemp juice are mixed in a ratio of 3:1 w/w, is described herein.

In some embodiments, a hemp, coffee fruit, and orange beverage comprising coffee fruit juice, hemp juice, and orange juice, wherein the hemp juice comprises juice from hemp inflorescences, is described herein.

In some embodiments, a hemp, coffee fruit, and orange beverage comprising coffee fruit juice, hemp juice, and orange juice, wherein the hemp juice comprises juice from hemp sun leaves, is described herein.

In some embodiments, a hemp, coffee fruit, and orange beverage comprising coffee fruit juice, hemp juice, and orange juice, wherein the orange juice comprises juice from a whole orange, is described herein.

In some embodiments, a hemp, coffee fruit, and orange beverage comprising coffee fruit juice, hemp juice, and orange juice, wherein the orange juice comprises juice from a peeled orange, is described herein.

In some embodiments, a beverage is prepared by contacting a water infusing powder with water, wherein the water infusing powder contains dehydrated coffee fruit pulp powder, dehydrated hemp pulp powder, and dehydrated orange pulp powder.

In some embodiments, a beverage is prepared by contacting a water-soluble dried powder with water, wherein the water-soluble dried powder contains dehydrated coffee fruit juice, dehydrated hemp juice, and dehydrated orange juice powder.

In some aspects, a hemp, coffee fruit, and orange beverage comprising coffee fruit juice, hemp juice, and orange juice, further comprising lemon juice and ginger is described herein.

In some embodiments, a water permeable container comprising a water infusing powder comprising dehydrated coffee fruit pulp powder, dehydrated hemp pulp powder, and dehydrated orange pulp powder, is described herein.

In some embodiments, a gel capsule comprising a water infusing powder comprising dehydrated coffee fruit pulp powder, dehydrated hemp pulp powder, and dehydrated orange pulp powder, is described herein.

In some embodiments, a gel capsule comprising a water-soluble powder comprising dehydrated coffee fruit juice, dehydrated hemp juice, and dehydrated orange juice powder, is described herein.

In some embodiments, a tablet comprising a water infusing powder comprising dehydrated coffee fruit pulp powder, dehydrated hemp pulp powder, and dehydrated orange pulp powder, is described herein.

In some embodiments, a tablet comprising a water-soluble powder comprising dehydrated coffee fruit juice, dehydrated hemp juice, and dehydrated orange juice powder, is described herein.

In some embodiments, a method of producing dehydrated orange pulp powder comprising the steps of providing pre-juiced orange tissue and exposing said tissue to ultrasonic waves, wherein the ultrasonic waves simultaneously pulverize and dehydrate the orange tissue, thereby producing dehydrated orange pulp powder, is described.

In some embodiments, a method of producing dehydrated orange pulp powder comprising the steps of providing pre-juiced orange tissue and exposing said tissue to ultrasonic waves, wherein the ultrasonic waves simultaneously pulverize and dehydrate the orange tissue, thereby producing dehydrated orange pulp powder, wherein the ultrasonic waves are at a frequency from about 1 kHz to about 20 kHz is described.

In some embodiments, a method of producing dehydrated orange pulp powder comprising the steps of providing pre-juiced orange tissue and exposing said tissue to ultrasonic waves, wherein the ultrasonic waves simultaneously pulverize and dehydrate the orange tissue, thereby producing dehydrated orange pulp powder, wherein the ultrasonic waves are emitted from an ultrapulverization machine is described.

In some embodiments, a method of producing dehydrated orange pulp powder comprising the steps of providing pre-juiced orange tissue and exposing said tissue to ultrasonic waves, wherein the ultrasonic waves simultaneously pulverize and dehydrate the orange tissue, thereby producing dehydrated orange pulp powder, further comprising the step of heating the dehydrated pulp powder in a microwave dehydrator, is described.

### DESCRIPTION OF THE DRAWINGS

FIG. 1A-B depicts various views of a coffee fruit. FIG. 1A depicts a transverse section of coffee fruit. FIG. 1B depicts a longitudinal section of coffee fruit. (a) corresponds to the disk (style remnants). (b) corresponds to epicarp (skin). (c) corresponds to the mesocarp (mucilage or pulp). (d) corresponds to the endocarp (parchment). (e) corresponds to the spermoderm (silver skin). (f) corresponds to the endosperm (coffee bean). (g) corresponds to the embryo.

FIG. 2 depicts coffee fruit of various stages of ripeness, including ripe (red or yellow) and unripe (green).

FIG. 3 depicts the anatomy of an orange. (a) corresponds to the wall. (b) corresponds to the rind. (c) corresponds to the pulp. (d) corresponds to the zest. (e) corresponds to a segment. (f) corresponds to the exocarp. (g) corresponds to the mesocarp. (h) corresponds to the juice sack. (i) corresponds to the seed. G) corresponds to the pip.

FIG. 4 depicts the anatomy of a Cannabis plant. The figure shows the male and female flowers (inflorescences), node, stem, and sun leaves. The female flowers include the pistil, calyx, cola, and trichomes.

FIG. 5 depicts the production process of a non-water-soluble powder comprising Cannabis (hemp), coffee fruit and/or oranges.

FIG. 6 depicts the production process of a water-soluble powder comprising Cannabis (hemp) juice, coffee fruit juice and/or orange juice.

### DETAILED DESCRIPTION OF THE INVENTION

### Definitions

As used herein, the verb “comprise” is used in this description and in the claims and its conjugations are used in its non-limiting sense to mean that items following the word are included, but items not specifically mentioned are not excluded.

As used herein, the term “about” refers to plus or minus 0.10% of the referenced number unless otherwise stated or otherwise evident by the context, and except where such a range would exceed 100% of a possible value, or fall below 0% of a possible value, such as less than 0% content of an ingredient, or more than 100% of the total contents of a composition. For example, reference to an absolute content of a particular cannabinoid of “about 1%” means that that terpene can be present at any amount ranging from 0.9% to 1.1% content by weight.

The disclosure provides beverages and methods of making beverages, which utilize plant parts from Cannabis hemp plants, coffee fruit plants, and oranges. As used herein, the term “plants” refers to plants in the genus of Cannabis, Citrus or Coffea and plants derived thereof. Plants may be produced via asexual reproduction, tissue culture, and via seed production.

The disclosure provides plant parts. As used herein, the term “plant part” refers to any part of a plant including but not limited to the embryo, shoot, root, stem, seed, stipule, leaf, petal, flower, inflorescence, bud, ovule, bract, trichome, branch, petiole, internode, bark, pubescence, tiller, rhizome, frond, blade, pollen, stamen, mesocarp, epicarp, endosperm, spermoderm, disk, embryo, and the like. The two main parts of plants grown in some sort of media, such as soil or vermiculite, are often referred to as the “above-ground” part, also often referred to as the “shoots”, and the “below-ground” part, also often referred to as the “roots”. Plant parts may also include certain extracts such as kief or hash, which includes Cannabis plant trichomes or glands. In some embodiments, plant part should also be interpreted as referring to individual cells from the plant.

The term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements is present, unless the context clearly requires that there is one and only one of the elements.

The International Code of Zoological Nomenclature defines rank, in the nomenclatural sense, as the level, for nomenclatural purposes, of a taxon in a taxonomic hierarchy (e.g., all families are for nomenclatural purposes at the same rank, which lies between superfamily and subfamily). While somewhat arbitrary, there are seven main ranks defined by the international nomenclature codes: kingdom, phylum/division, class, order, family, genus, and species.

The disclosure provides samples. As used herein, the term “sample” includes a sample from a plant, a plant part, a plant cell, or from a transmission vector, or a soil, water or air sample. Additional examples of samples include coffee fruit and hemp.

This disclosure utilizes “coffee fruit.” As used herein, the term “coffee fruit” is exchangeable with “coffee cherry.” “Coffee fruit” refers to any part of a coffee fruit, including the skin, pulp, mucilage, parchment, bean, endocarp, embryo. In some embodiments, the disclosure utilizes “whole coffee fruit,” which includes all parts of the coffee fruit. In some embodiments, the disclosure utilizes “de-beaned coffee fruit.” Wherever “de-beaned coffee fruit” is used herein, a person of ordinary skill in the art would understand that entire coffee fruit can be substituted.

In some embodiments, this disclosure utilizes oranges. As used herein, the term “orange” refers to any part of an orange, including wall, rind, pulp, zest, segment, exocarp, mesocarp, seed, and pip. In some embodiments, the present disclosure utilizes “whole oranges,” which includes all parts of the orange. In some embodiments, the present disclosure utilizes “peeled oranges,” which refers to the orange without its rind. In some embodiments, the present disclosure utilizes “non-juiced oranges”, which refers to oranges that have not been juiced, with or without their pericarps. In some embodiments, the present disclosure utilizes “juiced oranges”, which refers to oranges that have been juiced, with or without their pericarps.

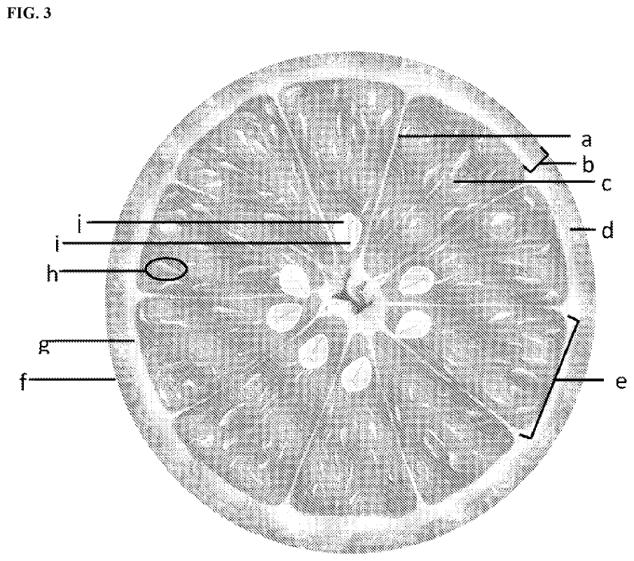

In some embodiments, the present disclosure refers to “orange pulp” as an ingredient for the compositions of the present disclosures. In this context, the term “orange pulp” is not limited to the “pulp” of an orange, as described in FIG. 3, but instead refers to orange pieces that have been cut/crushed/milled, or otherwise made into a pulpy texture. Thus, in the context of ingredients in the water infusing or water-soluble powders of the present disclosure, the term “orange pulp” can comprise other parts of the orange that have been broken up into small pieces (e.g., portions of the skin, or seeds).

In some embodiments, the present disclosure refers to a “dehydrated orange pulp powder” as a stand-alone product, with uses as a flavorant, dietary supplement, and anti-oxidant. In this context, the term “dehydrated orange pulp powder” refers to juiced oranges that have been dehydrated and pulverized using ultrasonic waves. The properties of this powder, as well as the methods of producing it are discussed in later sections of this disclosure.

In some embodiments, the present disclosure refers to hemp plants, hemp plant parts, hemp pulp, and hemp juice. Persons having skill in the art will recognize that any Cannabis plant is compatible with the inventions of the present disclosure, and that any reference to hemp should be understood to also refer to Cannabis more generally. Thus, in some embodiments, the water infusible compositions, water-soluble powders, and juices of the present disclosure can be made with hemp or with medicinal or recreational Cannabis.

In some embodiments, the present disclosure refers to inflorescences from a Cannabis, comprising particular cannabinoid and terpene contents (e.g., inflorescences comprising no more than 0.3% THC). In some embodiments, Cannabis inflorescences, such as hemp inflorescences, described as having cannabinoid content, are female inflorescences. In some embodiments, the inflorescences are grown “sinsemilla,” in the absence of male plants to avoid pollination and seed formation. Thus, in some embodiments, the female inflorescences of the present disclosure are seedless, and in many cases, un-pollinated. The term “inflorescence” and “flower” are used interchangeably throughout this document.

In some embodiments, the present disclosure teaches Cannabis plants, which are an annual, dioecious, flowering herb. Its leaves are typically palmately compound or digitate, with serrated leaflets. Cannabis normally has imperfect flowers, with staminate “male” and pistillate “female” flowers occurring on separate plants. It is not unusual, however, for individual plants to separately bear both male and female flowers (i.e., have monoecious plants). Although monoecious plants are often referred to as “hermaphrodites,” true hermaphrodites (which are less common in Cannabis) bear staminate and pistillate structures on individual flowers, whereas monoecious plants bear male and female flowers at different locations on the same plant.

As used herein, a “dioecious” plant refers to a plant having either only male flowers (androecious) or female flowers (gynoecious).

As used herein, a “monoecious” plant is a plant having both male and female or bisexual flowers, or both female and male or bisexual flowers. Plants bearing separate flowers of both sexes at the same time are called simultaneously or synchronously monoecious. Plants bearing flowers of one sex at one time are called consecutively monoecious.

Unless otherwise noted, references to cannabinoids in a plant, plant part, extract, or composition of the present disclosure should be understood as references to both the acidic and decarboxylated versions of the compound e.g., potential cannabidiol (CBD). For example, references to high CBD contents of a Cannabis plant in this disclosure should be understood as references to the combined CBD and CBDA content, accounting for weight loss during decarboxylation.

This disclosure provides beverages. As used herein, a beverage is any liquid that is suitable for human consumption. Beverages envisioned by the instant disclosure include, but are not limited to: In some embodiments, the present disclosure teaches a variety of beverage formulations comprising the water infusing or water-dissolvable powders of the present disclosure. Non-limiting examples of beverage formulations include frozen beverages, iced beverages, hot beverages, cold brews, seltzers, alcoholic beverages, non-alcoholic beverages, caffeinated beverages, decaffeinated beverages, carbonated beverages, non-carbonated beverages, soft drinks, cocktails, cocoas, ciders, beers, wines, teas, coffees, juices, energy drinks, mocktails, milkshakes, smoothies, tonic waters, liquors, and sodas.

The disclosure provides beverage ingredients. As used herein, a “beverage ingredient” refers to any ingredient necessary to make the components of a beverage. Non-limiting examples of beverage ingredients include coffee fruit, stabilizers, coating agents, hemp, flavorings, thickeners, anti-microbials, alcohol, and water.

In some embodiments of the disclosure, coffee fruit and hemp fruit are utilized to make a beverage. The beverage is made in a beverage container. As used herein, a beverage container refers to an object that can be used to hold or transport a liquid. Non-limiting examples of beverage containers include cups, bowls, jars, tubs, boxes, and bins.

As used herein, the term “water infusing powder” or “water-soluble powder” can comprise solid particles of a range of sizes and shapes. In some embodiments, the powder contains particles wherein the particle's longest dimension is less than about 4 cm, less than about 3.5 cm, less than about 3 cm, less than about 2.5 cm, less than about 2 cm, less than about 1 cm, less than about 0.5 cm, less than about 0.1 cm, less than about 9500, less than about 9000 microns, less than about 8500 microns, less than about 8000 microns, less than about 7500 microns, less than about 7000 microns, less than about 6500 microns, less than about 6000 microns, less than about 5500 microns, less than about 5000 microns, less than about 4500 microns, less than about 4000 microns, less than about 3500 microns, less than about 3000 microns, less than about 2500 microns, less than about 2000 microns, less than about 1500 microns, less than about 1000 microns, less than about 950 microns, less than about 900 microns, less than about 850 microns, less than about 800 microns, less than about 750 microns, less than about 700 microns, less than about 650 microns, less than about 600 microns, less than about 550 microns, less than about 500 microns, less than about 450 microns, less than about 400 microns, less than about 350 microns, less than about 300 microns, less than about 250 microns, less than about 200 microns, less than about 150 microns, less than about 100 microns, less than about 50 microns, less than about 40 microns, less than about 30 microns, less than about 20 microns, less than about 10 microns, or less than about 5 microns. In some embodiments, the powder contains particles which have dimensions based on the mesh size of the Fitzmill utilized to produce the powder. In some embodiments, the mesh size is between 5 mesh and 635 mesh. In some embodiments, the mesh size is about 5 mesh, or about 10 mesh, or about 15 mesh, or about 20 mesh, 25 mesh, or about 30 mesh, or about 35 mesh, or about 40 mesh, or about 45 mesh, or about 50 mesh, or about 55 mesh, or about 60 mesh, or about 65 mesh, or about 70 mesh, or about 75 mesh, or about 80 mesh, or about 85 mesh, or about 90 mesh, or about 95 mesh, or about 100 mesh, or about 105 mesh, or about 110 mesh, or about 115 mesh, or about 125 mesh, or about 135 mesh, or about 145 mesh, or about 155 mesh, or about 165 mesh, or about 175 mesh, or about 185 mesh, or about 195 mesh, or about 205 mesh, or about 215 mesh, or about 225 mesh, or about 235 mesh, or about 245 mesh, or about 255 mesh, or about 265 mesh, or about 275 mesh, or about 285 mesh, or about 295 mesh, or about 305 mesh, or about 315 mesh, or about 325 mesh, or about 335 mesh, or about 345 mesh, or about 355 mesh, or about 365 mesh, or about 375 mesh, or about 385 mesh, or about 395 mesh, or about 405 mesh, or about 415 mesh, or about 425 mesh, or about 435 mesh, or about 445 mesh, or about 455 mesh, or about 465 mesh, or about 475 mesh, or about 485 mesh, or about 495 mesh, or about 505 mesh, or about 515 mesh, or about 525 mesh, or about 535 mesh, or about 545 mesh, or about 555 mesh, or about 565 mesh, or about 575 mesh, or about 585 mesh, or about 595 mesh, or about 605 mesh, or about 615 mesh, or about 625 mesh, or about 635 mesh.

In some embodiments of the disclosure, a water infusing powder is utilized as a beverage component. As utilized herein, a water infusing powder is a composition that can be steeped in water or other solvent to impart that solvent with flavor and/or other nutrients (e.g., similar to steeping tea, or coffee in a French press). In some embodiments, the water infusing powders of the present disclosure are insoluble in water. Thus in some embodiments, the water infusing powders are less than 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% soluble in water. In some embodiments of the disclosure, a water-soluble powder is utilized as a beverage component. As used herein, a water-soluble powder is able to be dissolved in water. In some embodiments, a water-soluble powder dissolves in coffee, milk, soda, juice, or vinegar. In some embodiments, the water-soluble powder is about 80% dissolved, or about 81% dissolved, or about 82% dissolved, or about 83% dissolved, or about 84% dissolved, or about 85% dissolved, or about 86% dissolved, or about 87% dissolved, or about 88% dissolved, or about 89% dissolved, or about 90% dissolved, or about 91% dissolved, or about 92% dissolved, or about 93% dissolved, or about 94% dissolved, or about 95% dissolved, or about 96% dissolved, or about 97% dissolved, or about 98% dissolved, or about 99% dissolved, or about 100% dissolved when placed in water.

As used herein, the term “maturity,” “harvest maturity,” or “floral maturity” refers to the developmental stage at which coffee fruit or hemp plant is harvested. Persons having skill in the art will recognize maturity based on the plant's morphologies. For instance, the color of the coffee fruit can be used to indicate maturity. As used herein, the stage of ripeness is generally reflected by the amount of green coloring that remains on the surface of the coffee cherries, with more green coloring indicating increasingly less ripe.

In some embodiments, periodic cannabinoid content (i.e., potency) tests throughout the development of the hemp plant, is used to ensure that harvest occurs at maturity. In some embodiments, the hemp plant is considered to be at harvest maturity after inflorescences develop but before the hemp plant reaches a THC content of 0.3%. The inflorescences begin to take on a “frosted” appearance, as trichomes develop on calyxes and lower portions of bracts. The color of trichomes can also be used to determine maturity. Trichomes when first developing on the plant look small and clear, but gradually enlarge, and progressively become “milkier” and opaque with continued maturation, finally displaying a desiccated appearance and amber color. In some embodiments, the ideal time period after planting for floral maturity of the variety is typically around 12-18 weeks after planting seeds in the field, depending on planting date, and the variety grown.

In some embodiments, growing conditions throughout the hemp plant or coffee fruit plant's life cycle, nutrient variations, and environmental factors can all influence the amount of time for plants to reach harvest maturity. The present disclosure uses the terms “maturity,” “harvest maturity,” and “floral maturity” interchangeably.

In some embodiments, the sugar concentration in coffee fruit juice is monitored using Brix measurements. Brix values are generally used to measure sugar concentration in a liquid, but they can also be used as a proxy for extract concentration: the higher the Brix value, the more concentrated the extract. One degree Brix (° Bx) is equivalent to about 1 gram of sucrose in 100 grams of solution. ° Bx measurements are traditionally used to indicate the sweetness of fruit, fruit juice, maple syrup, and honey, among others. Brix measurements are monitored as described elsewhere in this disclosure.

Coffee Fruit and Hemp Products

In some embodiments, the present disclosure teaches products made from Cannabis (hemp), coffee fruit and/or oranges. Cannabis juice has potential health benefits but has a taste that is not widely accepted by consumers. The health benefits of Cannabis derive from compounds such as cannabinoids, terpenoids, and flavonoids, which are utilized to relieve chronic pain, to improve lung capacity, to stimulate weight loss, to regulate and/or prevent diabetes, to stabilize moods, to treat autism, to treat depression, to regulate seizures, to heal broken bones, to improve concentration and/or focus, to treat glaucoma, to alleviate anxiety, to slow development of Alzheimer's disease, to alleviate the pain associated with arthritis and/or multiple sclerosis, to treat posttraumatic stress disorder symptoms, to treat inflammatory bowel diseases, to combat alcoholism, and to reduce the tremors associated with Parkinson's Disease.

Coffee fruit is usually a discarded side product of coffee production. The juice from the pulp however has a pleasant flavor and has many beneficial properties. For example, coffee fruit contains compounds, such as antioxidants, which have a variety of health benefits. Non-limiting examples of compounds within coffee fruit include chlorogenic acids of various types, such as caffeoylquinic acids (CQA), dicaffeoylquinic acids (diCQA), feruloylquinic acids (FQA), p-coumaroylquinic acids (pCoQA), caffeoylferuloylquinic acids (CFAQ), diferuloylquinic acids, dimethoxycinamoylquinic acids, caffeoyl-dimethoxycinamoylquinic acids, feruloyl dimethoxycinamoylquinic acids, prodelphinidins, procyanidins, trigonelline, lignins, tannins, hydroxycinnamic acids, coffee saccharides, caffeic acid, quinic acid, ferulic acid, anthocyanins, and proanthocyanidins. Chlorogenic acids (CGAS) are a family of esters formed between certain hydroxycinnamic acids and quinic acid. Any such ester is included among the antioxidants and other beneficial compounds noted above.

Oranges contain many essential vitamins and nutrients, including vitamin C, vitamin A, thiamin, folate, potassium, fiber, proteins, copper, magnesium, flavonoids, hesperidin, and a variety of other trace vitamins and minerals. In some embodiments, orange juice is added to the products of the disclosure. In some embodiments, orange pulp is added to the products of the disclosure. Orange pulp is a typically discarded byproduct generated during orange juice production. In some embodiments, orange peel or orange zest is added to the products of the disclosure. Orange peel or orange zest contains provitamin A, fiber, folate, riboflavin, thiamine, vitamin B6, calcium, and polyphenols. In some embodiments, orange peels are utilized for their anti-inflammatory properties, anti-allergic characteristics, oral health benefits, ability to lower high blood pressure and cholesterol, ability to boost the immune system, anti-cancer activity, ability to improve the respiratory system, ability to improve the digestive system, and ability to stimulate weight loss.

Combining hemp, coffee fruit, and/or oranges results in a product that is both healthy, and unexpectedly desired among consumers. The present disclosure teaches a variety of hemp, coffee fruit, and/or orange products, including i) a water infusible powder comprising coffee fruit pulp and hemp pulp, ii) a water infusible powder comprising hemp pulp, and orange pulp, iii) a water infusible powder comprising coffee fruit pulp, hemp pulp, and orange pulp, iv) a water-soluble powder comprising hemp juice and coffee fruit juice, v) a water-soluble powder comprising hemp juice and orange juice, vi) a water-soluble powder comprising coffee fruit juice, hemp juice, and orange juice, vii) a mixture of hemp juice and coffee fruit juice, viii) a mixture of hemp juice and orange juice, and ix) a mixture of hemp juice, coffee fruit juice, and orange juice. The present disclosure also teaches beverages, which are prepared using the water infusible powder, water-soluble powder. Other products derived from the water-soluble and water infusible powders are also disclosed.

Cannabis (Hemp)

In some embodiments, the present disclosure teaches compositions comprising tissue and/or juice from a Cannabis plant Cannabis is a genus of flowering plants that includes at least three species, Cannabis sativa, Cannabis indica, and Cannabis ruderalis as determined by plant phenotypes and secondary metabolite profiles. In some embodiments, Cannabis can be used for recreational purposes (i.e., comprising greater than 0.3% THC). In some embodiments, the compositions and methods of the present disclosure utilize plant parts from medicinal or recreational Cannabis. In some embodiments, the compositions and methods of the present disclosure utilize plant parts from hemp. Hemp, also known as industrial hemp, is a type of Cannabis plant grown specifically for the industrial uses of its derived products. In the United States, Cannabis is classified as hemp if it accumulates no more than three-tenths of one percent (i.e., 0.3%) concentration of tetrahydrocannabinol (THC) at harvest maturity. Extracts produced from industrial hemp have no psychoactive effects, due to their low THC content.

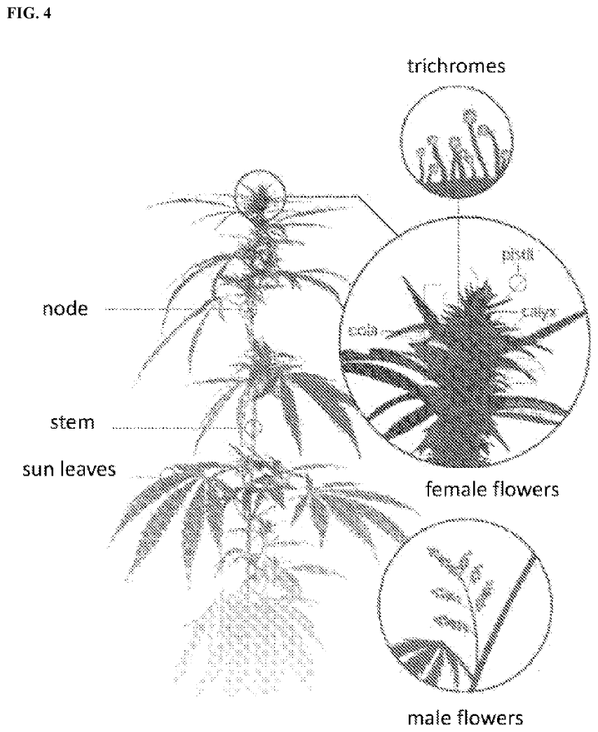

In some embodiments the Cannabis (hemp) is harvested after formation of flowers, but before senescence. In some embodiments the Cannabis (hemp) is harvested at harvest maturity. In some embodiments the Cannabis (hemp) is harvested after formation of flowers, but before the THC content of any portion of the plant exceeds 0.3% THC. The structure of the Cannabis plant is shown in FIG. 4.

In some embodiments, the entire hemp plant is harvested and processed at the same time. In other embodiments, the present disclosure teaches partial harvests, in which only the desired portions of the plants are harvested. For example, in some embodiments, the present disclosure teaches partial harvest of only portions of the leaf tissue, permitting the plant to continue developing. In some embodiments, the present disclosure teaches harvesting selected inflorescences, permitting the remaining inflorescences to continue to mature. Thus, in some embodiments, a single Cannabis plant can be harvested 1, 2, 3, 5, 6, 7, 8, 9, or 10 or more times for use in making the compositions of the present disclosure.

Coffee Fruit

Coffea is a genus of the family Rubiaceae. Coffea species are shrubs or small trees native to tropical and southern Africa and tropical Asia. As of the writing of this application, the only two states in the United States that grow coffee plants commercially are Hawaii and California.

There are over 120 species of Coffea. The two most popular are Coffea arabica (commonly known simply as “Arabica”), which accounts for 60-80% of the world's coffee production, and Coffea canephora (known as “Robusta”), which accounts for about 20-40%. In some embodiments, the present disclosure teaches use of any species of the coffee plant, including the species of Robusta (Robusta), Arabica (Arabica), Coffea liberia, C. excelsa, C. stenophylla, C. mauritiana. and C. racemosa.

Coffee plants produce edible red or purple fruits, sometimes referred to as “cherries”, which are described either as epigynous berries or as indehiscent drupes. The coffee fruit is best known for its two seeds, called “coffee beans.”

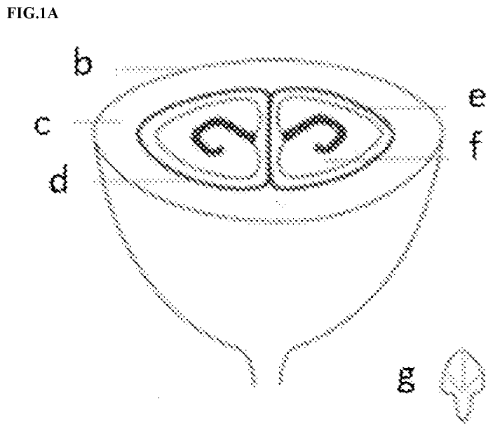

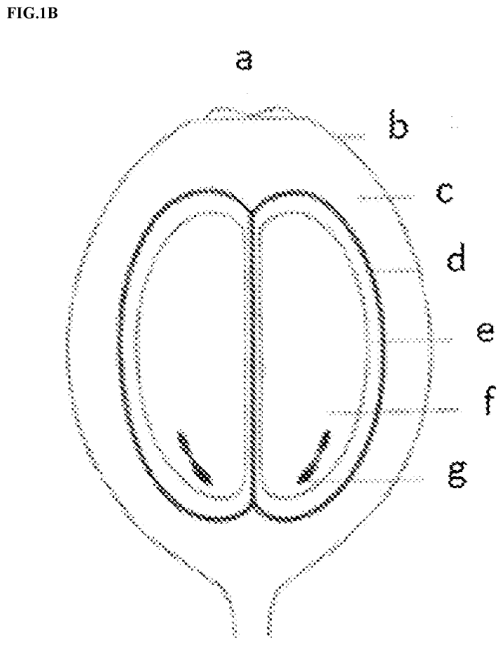

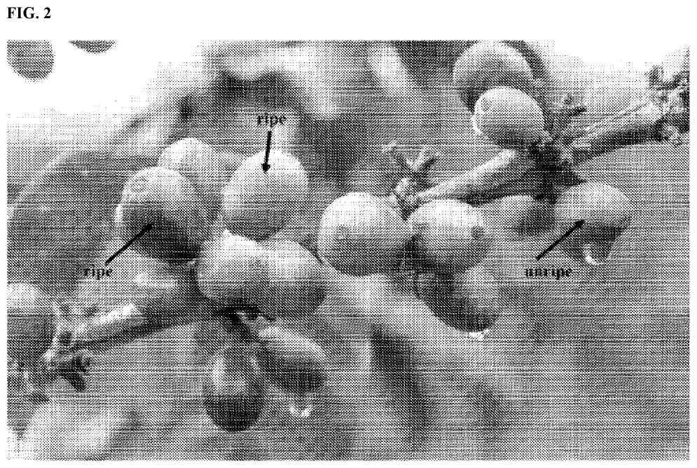

The coffee cherry is made up of the following layers (from the outside in): skin, pulp, mucilage, parchment and bean (FIG. 1A and FIG. 1B). The skin, also referred to as the epicarp or exocarp, is a monocellular layer covered with a waxy substance ensuring protection of the fruit. The mesocarp includes the pulp and the mucilage. The pulp is the fleshy outer layer of the mesocarp, directly beneath the skin, which during processing can be removed with a pulping machine. The mucilage is the slimy layer found between the pulp and parchment, adhering to the parchment inside a coffee cherry. It is generally not removed by pulping. Mucilage is not present in unripe coffee fruit and disappears in overripe coffee. Thus, in some embodiments, coffee fruit flesh or coffee fruit pulp in the present disclosure refers to the mesocarp of the coffee cherry, without the mucilage.

In some embodiments, persons having skill in the art will recognize the appropriate time to harvest the coffee cherries to maximize the presence of beneficial compounds and/or antioxidants. In some embodiments, the coffee cherries utilized in the present disclosure are harvested at harvest maturity. In some embodiments, the coffee cherries utilized in the present disclosure are harvested when the surface of the coffee cherry is less than about 50%, about 40%, about 30%, about 20%, about 10%, about 5%, or less than about 1% green. In some embodiments, the coffee cherries utilized in the present disclosure are ripe, that is, red, purple, dark purple, or yellow in color.

Oranges

Citrus is a genus of the family Rutaceae. Citrus species are shrubs or small trees native to China. As of the writing of this application, oranges are produced worldwide with 70% of the world orange production taking place in the Northern Hemisphere.

The two most common categories of oranges are the sweet orange and the bitter orange. Sweet oranges belong to Citrus sinensis, and bitter oranges belong to the family Citrus aurantium. In some embodiments, the present disclosure teaches use of a bitter orange, a sweet orange, or both. In some embodiments, the present disclosure teaches use of any species of orange, including the species of Citrus sinensis, Citrus aurantium, Citrus bergamia Risso, and Citrus reticulate. The orange is a citrus fruit that is made up of the following components, the wall, rind, pulp, zest (also called flavedo), segment, exocarp, mesocarp, juice sack, pip, and seed as shown in FIG. 3.

Products of the Present Disclosure, and Methods of Producing Same

Water Infusing Powder Comprising Dehydrated Hemp Pulp, Dehydrated Coffee Fruit Pulp, and/or Dehydrated Orange Pulp

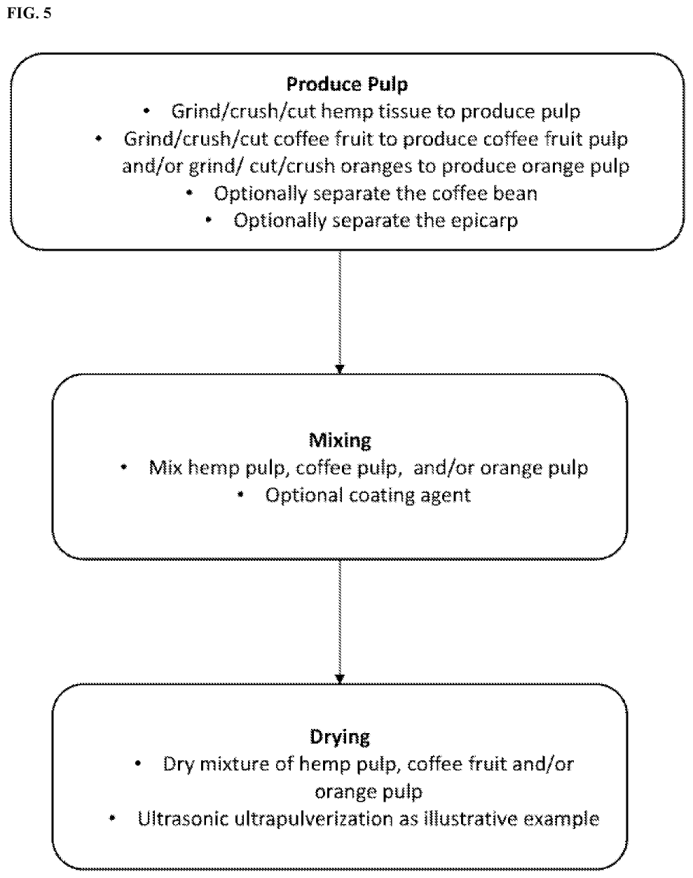

In some embodiments, the present disclosure provides for a water-infusing powder comprising hemp pulp, coffee fruit pulp and/or orange pulp. An illustrative method for producing the water-infusing powders of the present disclosure is outlined in FIG. 5.

Produce Pulp

In some embodiments, coffee fruit is first ground/crushed/cut or otherwise broken up to obtain a coffee fruit pulp. In some embodiments, the coffee fruit pulp is produced using a fruit crusher or grinder. In some embodiments, the coffee fruit pulp is produced in a mill. In some embodiments, the coffee fruit pulp is processed via wet milling.

In some embodiments, the present disclosure teaches making coffee fruit pulp from the entire fruit. In some embodiments, the present disclosure teaches making coffee fruit pulp from the fruit without the skin (epicarp). In some embodiments, the present disclosure teaches that coffee fruit pulp that includes the coffee bean results in a product containing caffeine. In some embodiments, the product contains caffeine at a level of about 0.01% w/w, about 0.02% w/w, about 0.03% w/w, about 0.04%, about 0.05% w/w, or about 0.010% w/w, or about 0.015% w/w, or about 0.020% w/w, or about 0.025% w/w, or about 0.030% w/w, or about 0.035% w/w, or about 0.040% w/w, or about 0.045% w/w, or about 0.050% w/w, or about 0.055% w/w, or about 0.060%, or about 0.065% w/w, or about 0.070% w/w, or about 0.075% w/w, or about 0.080% w/w, or about 0.085% w/w, or about 0.090% w/w, or about 0.095% w/w, or about 0.100% w/w. In some embodiments, the product contains about 30-250 mg of caffeine per eight fluid ounces. In some embodiments, the product contains from about 30 mg to about 40 mg of caffeine per eight fluid ounces. In some embodiments, the product contains from about 30 mg to about 50 mg of caffeine per eight fluid ounces. In some embodiments, the product contains from about 80 mg to about 100 mg of caffeine per eight fluid ounces. In some embodiments, the product contains from about 40 mg to about 250 mg of caffeine per eight fluid ounces. In some embodiments, the product contains about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 240 mg, about 245 mg, or about 250 mg of caffeine per eight fluid ounces of product. In some embodiments, the present disclosure teaches making the coffee fruit pulp from a de-skinned and debeaned fruit (i.e., only using the mescarp). In some embodiments, the present disclosure teaches that coffee fruit pulp that does not include the coffee bean results in a product with no measurable levels of caffeine. In some embodiments, the present disclosure uses a wet milling method in which harvested coffee cherries are fed into a pulping machine that separates the bean from the coffee fruit.

In embodiments, methods of the present disclosure involve grinding of the hemp, coffee fruit, and/or orange. In embodiments, grinding can be done using any method or machinery known in the art. For example, a Fitzpatrick Mill (Fitzmill) (Fitzpatrick Company. Elmhurst, Ind.) or HammerMill (Meadows Mills, Inc., North Wilkesboro, N.C.) can be used to grind the coffee cherries into powder per customer specifications. In some embodiments, the grinding step can be done using oxygen-free conditions.

In some embodiments, the present disclosure teaches orange pulp. In some embodiments, an orange pulp is made from the fruit without the peel. In some embodiments, the present disclosure teaches making orange pulp from an orange with the peel. In some embodiments, the present disclosure teaches making orange pulp from a juiced orange. In some embodiments, the present disclosure teaches making orange pulp from an orange fruit that has not been juiced. In some embodiments, the present disclosure teaches making orange pulp from a juiced orange without the peel. In some embodiments, the present disclosure teaches making orange pulp from a juiced orange with the peel. In some embodiments, the present disclosure teaches making orange pulp from an orange without the peel that has not been juiced. In some embodiments, the present disclosure teaches making orange pulp from an orange with the peel that has not been juiced. In some embodiments, peeling can be performed using any method or machinery known in the art. In some embodiments, peeling is performed using a swivel peeler, a Y peeler, a serrated peeler, a julienne peeler, a Lancashire peeler, a speed peeler, a mechanical peeler, or an automatic peeler. In some embodiments, the orange pulp is separated from a juiced orange (e.g., a juiced orange rind). In some embodiments the orange pulp is separated from orange juice (e.g., filtered or centrifuged from orange juice containing pulp). In some embodiments, juicing can be performed using any method or machinery known in the art.

In some embodiments, Cannabis (hemp) plant tissue is first ground/crushed/cut or otherwise broken up to obtain a Cannabis (hemp) pulp. In some embodiments, the Cannabis (hemp) pulp is produced using a fruit crusher or grinder. In some embodiments, the Cannabis (hemp) pulp is produced in a mill. In some embodiments, the coffee fruit pulp is processed via wet milling.

In some embodiments, the present disclosure provides pulp from Cannabis. In embodiments, the methods of the disclosure utilize pulp from hemp. In some embodiments, the present disclosure teaches making Cannabis pulp from any portion of a Cannabis plant. In some embodiments, the present disclosure teaches making Cannabis pulp from any above-ground portion of the Cannabis plant. In some embodiments, the present disclosure teaches making Cannabis pulp from any above-ground, de-stemmed portion of the Cannabis plant (e.g., all leafy green tissue and flowers). In some embodiments, the present disclosure teaches making Cannabis pulp from inflorescences of the plant.

Mixing

In some embodiments, the coffee fruit (with or without the bean and/or the skin) and the Cannabis (hemp) pulp, are mixed. In some embodiments, the coffee fruit (with or without the bean and/or the skin) and/or the Cannabis (hemp) pulp are mixed with orange pulp. In some embodiments, the Cannabis (hemp) pulp and the orange fruit pulp are mixed. In some embodiments, the pulps are mixed in any convenient container or surface, including but not limited to tabletops, plates, bowls, or mixing tanks. In some embodiments the mixing surface container is part of a larger machinery, such as a mill, oven, dehydrator, or ultrapulverization machine.

In some embodiments, the Cannabis (hemp) pulp and coffee fruit pulp and/or orange pulp are mixed with one or more coating agent(s). In some embodiments, water is also added to facilitate mixing of the ingredients. In some embodiments, the Cannabis (hemp) pulp and coffee fruit pulp and/or orange pulp are not mixed with a coating agent.

In embodiments, the coating agent is a preservative and/or anti-oxidizing agent. In some embodiments, the preservative coating agent inhibits the degradation of the antioxidants and other beneficial compounds in the coffee fruit, for example, by preventing oxidation or enzymatic degradation. For example, phenolic compounds in the coffee fruit can be oxidized by an enzyme called polyphenol oxidase (PPO). This process is evidenced by the browning of the coffee fruit.

Also, coffee fruit enzymes can convert free phenols and amino acids to quinic acid. In some embodiments, the coating agent retards these degradative processes and denatures the enzymes that promote oxidation, thus preserving higher amounts of antioxidants, amino acids, and free phenols.

In some embodiments the coating agent may comprise any known anti-oxidant. In some embodiments, the coating agent is selected from the group consisting of: ascorbic acid, citric acid, acetic acid, benzoic acid, sulfur dioxide, sulfites such as potassium sulfite, and any combinations thereof. In embodiments, the coating agent can include a combination of ascorbic acid and citric acid in water or another suitable solvent. In some embodiments, the water is provided by the pulp. In some embodiments, the coating agent is dehydrated orange pulp powder.

Persons having skill in the art will recognize the appropriate amount of coating agent necessary to avoid oxidation and degradation of the pulp. In some embodiments, the coating agent can be used in an amount of up to or about 1 gram per gallon, 2 grams per gallon, 3 grams per gallon, 4 grams per gallon, 5 grams per gallon, 6 grams per gallon, 7 grams per gallon, 8 grams per gallon, 9 grams per gallon, 10 grams per gallon, 11 grams per gallon, 12 grams per gallon, 13 grams per gallon; 14 grams per gallon, 15 grams per gallon, 20 grams per gallon of pulp mixture, including any ranges and subranges therebetween.

In some embodiments, powders, such as a food-grade powder of ascorbic acid can be used as a coating agent. For example, in some embodiments, the ascorbic acid can be present in about 15 grams food grade powder per gallon of pulp, including any ranges and subranges therebetween. In some embodiments, the coating agent is citric acid. In some embodiments, the citric acid can be used in an amount of up to or about 0.5 grams per gallon of pulp mixture, 1 gram per gallon, 2 grams per gallon, 3 grams per gallon, 4 grams per gallon, 5 grams per gallon, 6 grams per gallon, 7 grams per gallon, 8 grams per gallon, 9 grams per gallon, 10 grams per gallon, 11 grams per gallon, 12 grams per gallon, 13 grams per gallon, 14 grams per gallon, 15 grams per gallon, 20 grams per gallon of pulp, including any ranges or subranges thereof.

In some embodiments, the coating agent is ascorbic acid. In some embodiments, the ascorbic acid can be used in an amount of up to or about 0.5 grams per gallon of pulp mixture, 1 gram per gallon, 2 grams per gallon, 3 grams per gallon, 4 grams per gallon, 5 grams per gallon, 6 grams per gallon, 7 grams per gallon, 8 grams per gallon, 9 grams per gallon, 10 grams per gallon, 11 grams per gallon, 12 grams per gallon, 13 grams per gallon, 14 grams per gallon, 15 grams per gallon, 20 grams per gallon of pulp mixture, including any ranges or subranges thereof. In some embodiments, 3 grams of ascorbic acid is added per gallon of pulp mixture. In some embodiments, 1 teaspoon (tsp) of ascorbic acid is added per gallon of pulp mixture.

In some embodiments, the coating agent is dehydrated orange pulp powder. In some embodiments, the orange can be used in an amount of up to or about 0.5 grams per gallon of pulp mixture, 1 gram per gallon, 2 grams per gallon, 3 grams per gallon, 4 grams per gallon, 5 grams per gallon, 6 grams per gallon, 7 grams per gallon, 8 grams per gallon, 9 grams per gallon, 10 grams per gallon, 11 grams per gallon, 12 grams per gallon, 13 grams per gallon, 14 grams per gallon, 15 grams per gallon, 20 grams per gallon of pulp mixture, including any ranges or subranges thereof.

In some embodiments, the Cannabis (hemp) pulp, coffee fruit pulp and/or the orange pulp are mixed to achieve a good blend between the ingredients. In some embodiments, the Cannabis (hemp) pulp, coffee fruit pulp and/or orange pulp are mixed to ensure that the pulps are coated in the coating agent. In some embodiments the mixing is done by hand. In other embodiments, the mixing is done via one or more machines.

Drying

In some embodiments, the Cannabis (hemp) pulp, coffee fruit pulp and/or orange pulp mixture are dried to produce a water-infusible powder. In embodiments, the pulp mixture is drained of excess water. In some embodiments the draining step comprises any form of separating water from solid material, including sieves, centrifuges, presses, shakers, etc. In some embodiments, the water is not drained.

Persons having skill in the art will be familiar with various industrial and commercial methods for drying food products. In some embodiments, the pulp mixture can be dried by any known drying/dehydrating technique.

In some embodiments, the pulp mixture is dehydrated using a method selected from the group consisting of ultrasound ultrapulverization or heating.

In some embodiments the pulp mixture is dehydrated using a food dehydrator. In some embodiments the pulp mixture is dehydrated using a microwave food dehydrator. A non-limiting list of commercial food dehydrators includes those described in the following U.S. patent applications and patents: US 2016/0360773, D252397, D270423, D744273, US 2015/0272199, U.S. Pat. Nos. 3,414,982, 6,085,442, D261142, U.S. Pat. Nos. 10,337,794, 5,423,249, US 2015/0257431, US 2009/0025248, D738671, US 2013/0326902, US 2018/0271122, U.S. Pat. Nos. 3,264,753, 4,536,643, 4,190,965, 9,877,502, D401468, U.S. Pat. No. 2,359,199, D691842, U.S. Pat. Nos. 5,878,508, 9,801,406, 5,974,957, US 2015/0101494, US 2013/0233185, US 2018/0106544, U.S. Pat. Nos. 4,534,118, 4,036,562, 10,378,821, US 2018/0306510, U.S. Pat. Nos. 5,437,108, 4,531,306, 2,414,642, each of which is hereby incorporated by reference.

In some embodiments, the present disclosure teaches that the drying/dehydration step must take place at a temperature of less than 130° F., 125° F., 120° F., 115° F., 110° F., 105° F., 100° F., 95° F., 90° F., 85° F., 80° F., 75° F., 70° F., 65° F., 60° F., 55° F., or 50° F., including all ranges and subranges therebetween. In some embodiments, the present disclosure teaches that the drying/dehydration step must take place a temperature between about 105° F. and 115° F. In some embodiments, the present disclosure teaches that the temperature of the drying/dehydration step must not exceed 130° F.

In some embodiments, the dehydration can be done for a period of time, for example at least about 1 hour, 2 hours, 3 hours, 3 hours, 5 hours, 6 hours, 7 hours, 8 hours, 9 hours, 10 hours, 11 hours, 12 hours, 13 hours, 13 hours, 15 hours, 16 hours, 17 hours, 18 hours, 19 hours, 20 hours, 23 hours, 38 hours or longer. For example, dehydration can be done at a temperature of about 130 to about 135° F., or about 135° F., for about 10-12 hours, or until a water content of about 5% or 6% is achieved. In some embodiments, the dehydration step can be carried out under oxygen-free conditions, for example by exposing the coffee cherries to an inert gas such as nitrogen during dehydration.

In some embodiments the pulp mixture is dehydrated using an ultrasound pulverization system. In some embodiments, the ultrasound pulverization system is a C-DC Grind & Dry Milling System (Enagon), a UIP500hd (Hielscher), a UIP1000hd (Heilscher), a UIP1500hd (Heilscher), a UIP2000hd (Heilscher), a UIP4000 (Heilscher), a UIP10000 (Heilscher), or a UIP16000 (Heilscher). In some embodiments, ultrasound pulverization pulverizes the beverage ingredients to a powder. In some embodiments, ultrasound pulverization is utilized to dry the Cannabis (hemp) pulp, coffee fruit pulp and/or orange pulp ingredients. In some embodiments, ultrasound pulverization pulverizes the beverage ingredients to a powder of defined particle sizes. In some embodiments, the ultrasound pulverizer uses sound energy frequencies from about 1 kHz to about 20 kHz.

In some embodiments, freeze drying is utilized to dehydrate components of the beverages of the disclosure. Freeze-drying is technology in which a substance is frozen as the substance is dried. In this type of process, the substance to be dried (which need not be a liquid) is first frozen so that the water contained in it is turned to ice. The ice is then removed by sublimation, which is the direct evaporation of the gas (water vapor) from the solid state (ice) without ever becoming liquid.

In some embodiments, the dehydrated pulp mixture is further placed in a microwave dehydrator to remove additional moisture and kill off mycotoxins and/or mold. The dehydrated pulp mixture thus becomes a water infusible powder.

In some embodiments, the dehydrated pulp mixture is comprising a moisture content of less than 15%, 14.5%, 14%, 13.5%, 13%, 12.5%, 12%, 11.5%, 11%, 10.5%, 10%, 9.5%, 9%, 8.5%, 8%, 7.5%, 7%, 6.5%, 6%, 5.5%, 5%, 4.5%, 4%, 3.5%, 3%, 2.5%, 2%, 1.5%, 1%, 0.5%, and any ranges and subranges therein. In some embodiments, the dehydrated pulp mixture comprises a moisture of 4%.

Packaging/Use

In embodiments, the resulting water infusible powder comprising the dehydrated hemp powder, coffee fruit and/or orange powder is added to a container that is permeable to water, while keeping insoluble pieces of solid material in the container. In some embodiments the container is a filter bag, infuser, tea basket, tea strainer, coffee filter, Keurig cup, colander, or similar device.

In some embodiments, the water infusible power in the water permeable container is contacted with water to produce a tea-like beverage from the dried coffee fruit pulp and Cannabis (hemp) pulp.

Persons having skill in the art will recognize that the water infusible powder compositions do not have to be created via the exact steps illustrated above. For example, in some embodiments, the Cannabis (hemp) pulp, coffee fruit pulp and/or orange pulp can be separately dehydrated, and then mixed after the drying step in some embodiments, the Cannabis (hemp) plant tissue, coffee fruit and/or orange can be mixed before being crushed/ground/cut (e.g., processed together). In some embodiments, the coating agent can be separately applied to the orange pulp and/or coffee fruit pulp and/or the Cannabis (hemp) pulp before mixing them as a pulp.

Water-Soluble Powder Comprising Hemp Juice, Coffee Fruit Juice, and/or Orange Juice

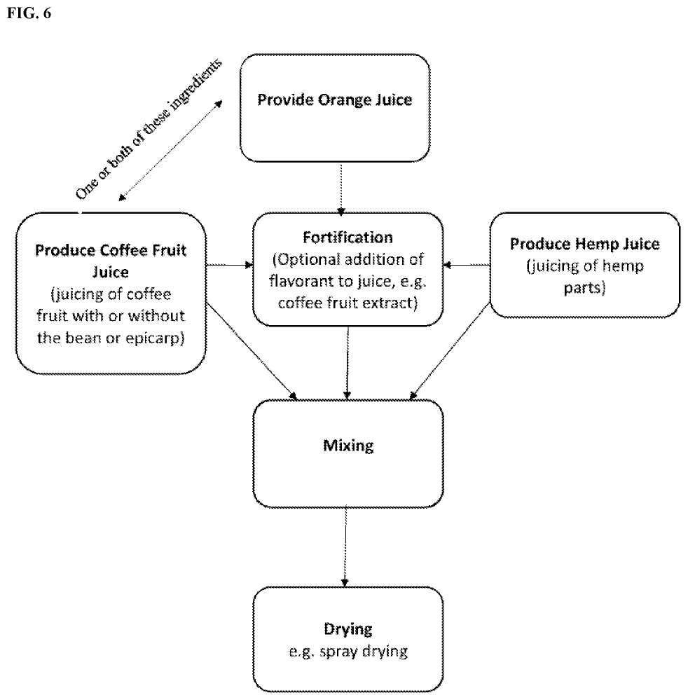

In some embodiments, the present disclosure provides for a water-soluble powder comprising hemp juice, coffee fruit juice and/or orange juice. An illustrative method for the production of the water-soluble powder is displayed in FIG. 6.

Coffee Fruit Juice Production

In some embodiments, the products and methods of the present disclosure comprise coffee fruit juice. In some embodiments, the present disclosure teaches making coffee fruit juice from the entire fruit. In some embodiments, the present disclosure teaches making coffee fruit juice from the fruit without the skin (epicarp). In some embodiments, the present disclosure teaches that coffee fruit juice that includes the coffee bean results in a product comprising higher levels of caffeine. In some embodiments, the present disclosure teaches making the coffee fruit juice from a de-skinned and debeaned fruit (i.e., only using the mesocarp). In some embodiments, the present disclosure uses a wet milling method in which harvested coffee cherries are fed into a juicing machine that separates the bean from the coffee fruit.

In some embodiments, coffee fruit juice is generated using any technique suitable for extracting juice from a fruit. In embodiments, coffee fruit are crushed to form coffee fruit juice. In some embodiments, any commercial or consumer grade juicer or press can be used. In some embodiments, centrifugal juicers, masticating juicers, or triturating juicers are utilized to generate coffee fruit juice. Non-limiting examples of juicers include the JE98XL juicer (Breville), the 67601 A Big Mouth Juice Extractor Electric Juicer (Hamilton Beach), the 800JEXL juicer (Breville), the J8004 Nutrition Center Commercial Masticating Juicer (Omega), the Avamix JE700 Continuous Feed Juice Extractor with Pulp Ejection, the Hamilton HCJ967 Electric commercial juicer, an Industrial Fruit Juicer Extruder Press, a good nature juicer machine, and the Waring 6001C Heavy-Duty Juice Extractor.

In some embodiments, coffee fruit juice with a defined sugar content is prepared. In some embodiments, a person of skill in the art will prepare a coffee fruit juice with a sugar content optimized for taste. In embodiments, the coffee fruit juice of the present disclosure contains a sugar content of about 0.1° Bx, 1° Bx, 2° Bx, 3° Bx, 4° Bx, 5° Bx, 6° Bx, 7° Bx, 8° Bx, 9° Bx, 10° Bx, 11° Bx, 12° Bx, 13° Bx, 14° Bx, or 15° Bx, including any ranges and subranges therein. In embodiments, the coffee fruit juice has a lower Brix value, for example less than about 10° Bx, 8 0Bx, 6° Bx, or 4° Bx or lower, or between about 0.1° Bx and 10° Bx, 1° Bx and 5° Bx, and 1° Bx and 3° Bx, or about 1.5° Bx to about 2.5° Bx. In some embodiments, the coffee fruit juice has a sugar content of between 3° Bx to 4° Bx.

In some embodiments, the coffee fruit juice is partially evaporated to obtain a coffee fruit juice with a higher sugar content. In some embodiments, the coffee fruit juice is evaporated by a ratio of up to about 5:1 v/v, about 10:1 v/v, about 15:1 v/v, about 20:1 v/v, about 25:1 v/v, about 35:1 v/v, about 40:1 v/v, about 45:1 v/v, about 50:1 v/v, about 60:1 v/v or higher (where the ratio indicates initial versus final volume). The coffee fruit juice can be concentrated to any desired level, from a relatively dilute extract to a very concentrated and viscous extract and even subjected to complete evaporation (for example less than about 10% moisture content, or about 5-6% moisture content or below) to provide a dry extract concentrate.

In some embodiments, the coffee fruit juice is diluted to obtain a coffee fruit juice with a lower sugar content. In some embodiments, the coffee fruit juice is diluted by a ratio of about 1:1 v/v, about 1:2 v/v, about 1:3 v/v, about 1:4 v/v, about 1:5 v/v, about 1:10 v/v, about 1:15 v/v, about 1:20 v/v, about 1:25 v/v, about 1:35 v/v, about 1:40 v/v, about 1:45 v/v, about 1:50 v/v, about 1:60 v/v or lower (where the ratio indicates initial versus final volume). The juice can be diluted to any desired level, from a relatively dilute extract to a very concentrated extract.

In some embodiments, the sugar content of the compositions of the disclosure are measured. In some embodiments, Brix measurements are performed by refractive index. In some embodiments, Brix measurements are performed by density. In some embodiments, an instrument is utilized to take Brix measurements. Non-limiting examples of instruments to measure Brix values include a hydrometer, a pycnometer, a refractometer, and a digital density meter. In embodiments, the sugar content of the compositions of the disclosure is measured using a “Brix Meter” or “Refractometer.”

Cannabis (Hemp) Juice Production

In some embodiments, the compositions of the present disclosure comprise hemp juice. In some embodiments, the present disclosure teaches making Cannabis juice from any portion of a Cannabis plant. In some embodiments, the present disclosure teaches making Cannabis juice from any above-ground portion of the Cannabis plant. In some embodiments, the present disclosure teaches making Cannabis juice from any above-ground, de-stemmed portion of the Cannabis plant (e.g., all leafy green tissue and flowers). In some embodiments, the present disclosure teaches making Cannabis juice from inflorescences of the plant.

In some embodiments, Cannabis (hemp) juice is generated using any technique suitable for extracting juice from a fruit. In embodiments, Cannabis tissue is crushed to form Cannabis juice. In some embodiments, any commercial or consumer grade juicer or press can be used, for example those disclosed above for use with coffee fruit. In some embodiments, hemp juice is prepared by cold pressing Cannabis (hemp). In embodiments, the Cannabis (hemp) plant (or harvested portion thereof) is pressed immediately after harvest without prior drying. In embodiments, the plant is cleaned from soil and contaminants before being pressed as described herein.

The Cannabis (hemp) juice of the present disclosure can be made with any technique or device capable of extracting liquid from plant tissue, including those listed in the coffee fruit juice section. In some embodiments, any commercial or consumer grade juicer or press can be used.

Orange Fruit Juice Production

In some embodiments, the products and methods of the present disclosure comprise orange juice. In some embodiments, the present disclosure teaches making orange juice from the entire fruit. In some embodiments, the present disclosure teaches making orange juice from the pulp of the fruit.

Persons having skill in the art will be aware of various methods of obtaining orange juice, including commercially purchasing said juice on the market. In some embodiments, orange fruit juice is generated using any technique suitable for extracting juice from a fruit, including those described in the coffee fruit section above.

Fortification

In some embodiments, the compositions of this disclosure are fortified. In some embodiments, the hemp fruit juice or coffee fruit juice are fortified individually, prior to mixing. In some embodiments, the mixture of hemp fruit juice and coffee fruit juice is fortified. In some embodiments, the compositions of this disclosure are fortified with one or more substances selected from the group consisting of an enriching agent, a sweetener, a stabilizer, a flavoring, an anti-microbial, a thickener, and a nutrient. In some embodiments, the compositions of the present disclosure are fortified with dehydrated orange juice powder or dehydrated orange pulp powder. Orange pulp and dehydrated orange pulp powder are prepared according to the methods described elsewhere in the disclosure. In some embodiments, the compositions of the present disclosure are fortified with coffee fruit extract.

In embodiments, coffee fruit extract is made by the method of (a) providing de-beaned coffee cherries, (b) optionally, freezing the coffee cherries at a temperature of less than about −30° F. to produce frozen de-beaned coffee cherries, (c) and thawing the frozen de-beaned coffee cherries to produce thawed de-beaned coffee cherries, and (d) extracting antioxidants from the thawed de-beaned coffee cherries in a heated aqueous solvent to produce a liquid coffee fruit extract. In some embodiments, the coffee fruit extract comprises a total phenolic acid concentration of at least 5% (w/v). In some instances, the coffee fruit extract comprises a total phenolic acid concentration of at least about 20% (w/w).

Further examples of enriching agents, sweeteners, stabilizers, flavorings, anti-microbials, and thickeners are described in the formulation section of this application.

Mixing

In some embodiments, the optionally fortified Cannabis (hemp) juice, optionally fortified coffee fruit juice (prepared with or without the bean and/or the skin) and/or the optionally fortified orange juice are mixed. In some embodiments, the juices are mixed in any convenient container or surface, including but not limited to tabletops, plates, bowls, mixing tanks. In some embodiments the mixing surface container is part of a larger machinery, such as a mill, oven, dehydrator, or spray-drying machine.

In some embodiments, coffee fruit juice and hemp juice are mixed in a 10:1 w/w, 9:1 w/w, 8:1 w/w, 7:1 w/w, 6:1 w/w, 5:1 w/w, 4:1 w/w, 3:1 w/w, 2:1 w/w, 1:2 w/w, 1:3 w/w, 1:4 w/w, 1:5 w/w, 1:6 w/w, 1:7 w/w, 1:8 w/w, 1:9 w/w, or 1:10 w/w ratio to form a mixture of coffee fruit juice and hemp juice. In some embodiments, orange fruit juice and hemp juice are mixed in a 10:1 w/w, 9:1 w/w, 8:1 w/w, 7:1 w/w, 6:1 w/w, 5:1 w/w, 4:1 w/w, 3:1 w/w, 2:1 w/w, 1:2 w/w, 1:3 w/w, 1:4 w/w, 1:5 w/w, 1:6 w/w, 1:7 w/w, 1:8 w/w, 1:9 w/w, or 1:10 w/w ratio to form a mixture of orange fruit juice and hemp juice. In some embodiments, compositions comprising coffee fruit juice, orange juice, and hemp juice are mixed in parts ranging from 1-10 for each ingredient.

In embodiments, the mixture of hemp juice, coffee fruit juice, and/or orange juice is consumed as a beverage, without any subsequent drying steps. In embodiments, the mixture of coffee fruit juice and hemp juice is dehydrated using any of the methods for dehydration herein to form a water-soluble powder. In embodiments, the juice mixture is dehydrated by spray-drying. In embodiments, the dehydrated juice mixture is further ground into finer particles.

Drying

In some embodiments, the hemp juice, coffee fruit juice and/or orange juice mixture are dried to produce a water-soluble powder. Persons having skill in the art will be familiar with various industrial and commercial methods for drying liquids. In some embodiments, the juice mixture can be dried by any known drying/dehydrating technique, including but not limited to: freeze drying, evaporation, boiling, and spray drying. In some embodiments, spray drying is utilized to dry the juice mixture. Spray drying is a process in which a homogeneous aqueous mixture of drug and the carrier is introduced via a nozzle (e.g., a two-fluid nozzle), spinning disc or an equivalent device into a hot gas stream to atomize the solution to form fine droplets. The aqueous mixture may be a solution, suspension, slurry, or the like. In some embodiments, the aqueous mixture is a solution. The solvent, generally water, rapidly evaporates from the droplets producing a powder. In some embodiments, the juices of the disclosure may be sprayed dried in conventional spray drying equipment from commercial suppliers, such as Buchi, Niro, Yamato Chemical Co., Okawara Kakoki Co., and the like, resulting in a substantially amorphous particulate product. In some embodiments, the spray drying process utilizes methods, such as rotary atomization, pressure atomization and two-fluid atomization. Non-limiting examples of the devices used in these processes include “Parubisu [phonetic rendering] Mini-Spray GA-32” and “Parubisu Spray Drier DL-41”, manufactured by Yamato Chemical Co., or “Spray Drier CL-8,” “Spray Drier L-8,” “Spray Drier FL-12,” “Spray Drier FL-16” or “Spray Drier FL-20,” manufactured by Okawara Kakoki Co., can be used for the method of spraying using rotary-disk atomizer.

In embodiments, the water-soluble juice powder can be distributed in containers, such as cans, boxes, bags, or small packets for individual use. In embodiments, the water-soluble coffee fruit juice, hemp juice powder, and dehydrated orange pulp powder can be distributed in containers, such as cans, boxes, bags, or small packets for individual use. In some embodiments, the water-soluble powder is consumed directly, either as a powder, or formulated into a pill, capsule, or other tablet. In some embodiments, the water-soluble powder is dissolved in water to form a juice-like beverage, which can then be consumed alone, or in combination with other ingredients.

Persons having skill in the art will recognize that the water-soluble powder compositions do not have to be created via the exact steps illustrated above. For example, in some embodiments, Cannabis (hemp) juice, the coffee fruit juice and/or orange juice can be separately dehydrated/dried, and then mixed after the drying step. In some embodiments, the coffee fruit and Cannabis (hemp) plant tissue can be mixed before being juiced (e.g., processed together). In some embodiments, the one or more optional fortifying agents can be separately applied to the orange juice, coffee fruit juice and/or the Cannabis (hemp) juice before mixing them, or after mixing them. In some embodiments, the one or more optional fortifying agents can be applied or to the dried powder composition, after the drying step.

Dehydrated Orange Pulp Powder

In some embodiments, the present disclosure provides for a dehydrated orange pulp powder. In some embodiments the dehydrated orange pulp powder can be used as a food ingredient, dietary supplement, and/or industrial food anti-oxidant.

In some embodiments, the dehydrated orange pulp powder is produced from pre-juiced oranges. In some embodiments, the dehydrated orange pulp powder is produced from pre juiced oranges, including the peel. In some embodiments, the dehydrated orange pulp powder is produced from the remaining pulp without the peel. In some embodiments, the dehydrated orange pulp powder is produced from pulp recovered (e.g., filtered or centrifuged) from orange juice.

The present disclosure teaches methods of producing dehydrated orange pulp powder, said method comprising the steps of: a) providing pre juiced orange tissue, and b) exposing said tissue to ultrasonic waves; wherein the ultrasonic waves simultaneously pulverize and dehydrate the orange tissue. In some embodiments, the ultrasonic waves are at a frequency from about 1 kHz to about 20 kHz. In some embodiments, the ultrasonic waves are administered via an ultrapulverization machine. Exemplary ultrapulverization machines are described throughout this disclosure. In some embodiments, the methods for producing dehydrated orange pulp powder comprise an additional drying step to further reduce the moisture content.

Without wishing to be bound by any one theory, it is believed that the dehydrated orange pulp powder of the present disclosure exhibits greater flavor and nutritional properties than other dehydrated orange products. Specifically, the dehydration and pulverization techniques produce a powder that is: i) more bioavailable, and/or ii) preserves greater quantities of vitamins and nutrients from the orange tissue than traditional dehydrated orange products. For example, in some embodiments, the dehydration and pulverization techniques of the present disclosure produce dehydrated orange pulp powder with smaller particle sizes that other traditional orange products. In some embodiments, the smaller particle size enhances the flavor of the dehydrated orange pulp powder by increasing the surface area of the particles. The greater surface area of the instantly disclosed powders increase the flavor that the powder imparts to drinks or food products by improving vitamin and nutrient solubility/availability. Indeed, in some embodiments, the dehydrated orange pulp powder of the present disclosure exhibits a stronger flavor when directly consumed (e.g., in a tablet or powder form) compared to traditional dehydrated orange products.

In some embodiments, the dehydrated orange pulp powder of the present disclosure will contain a higher content of vitamins, nutrients and antioxidants than traditional dehydrated orange products. In some embodiments, the rapid dehydration using ultrasonic waves avoids the vitamin, nutrient, and/or antioxidant decay associated with other forms of dehydration.

In some embodiments, the present disclosure teaches dehydrated orange pulp powder. In some embodiments the dehydrated orange pulp powder has an average particle size of 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 740 μm, 750 μm, 760 μm, 770 μm, 780 μm, 790 μm, 800 μm, 810 μm, 820 μm, 830 μm, 840 μm, 850 μm, 860 μm, 870 μm, 880 μm, 890 μm, 900 μm, 910 μm, 920 μm, 930 μm, 940 μm, 950 μm, 960 μm, 970 μm, 980 μm, 990 μm, or 1000 μm, or 2000 μm, or 3000 μm, or 4000 μm, or 5000 μm, including all ranges and subranges therebetween.

In some embodiments, the present disclosure teaches dehydrated orange pulp powder with a moisture content of less than 0.5% w/w, 1% w/w, 1.5% w/w, 2% w/w, 2.5% w/w, 3% w/w, 3.5% w/w, 4% w/w, 4.5% w/w, 5% w/w, 5.5% w/w, 6% w/w, 6.5% w/w, 7% w/w, 7.5% w/w, 8% w/w, 8.5% w/w, 9% w/w, 9.5% w/w, or 10% w/w.

Formulations

In some embodiments, Cannabis (hemp), coffee fruit, and/or orange compositions of the present disclosure may be further formulated to enhance flavor, increase shelf life, enhance health benefits, or to generate new modes of consumption/delivery. Formulating agents include enriching agents, sweeteners, stabilizers, flavorings, anti-microbials, and thickeners. The formulating agents can be added at any step during the process of creation of the water-infusing powders, water-soluble powders, beverages, and other products of the present disclosure. In some embodiments, the formulating agents are added before mixing. In some embodiments, the formulating agents are added before drying. In some embodiments, the formulating agents are added after drying. In some embodiments, the formulating agents are added to beverages of the disclosure.

Water and Water Substitutes

In some embodiments, the compositions of the present disclosure and methods of the present disclosure utilize water. In some embodiments, water is utilized for preparation of a beverage comprising the water infusing or water-soluble powder of the disclosure. In some embodiments, tap water, mineral water, spring water, glacier water, sparkling water, distilled water, purified water, flavored water, infused water, alkaline water, or well water, can be utilized in the present disclosure.

In some embodiments, the beverages of the present disclosure utilize water-containing liquids, such as juice, milk, or soda. In some embodiments, the Cannabis (hemp) and coffee fruit juice compositions of the present disclosure (whether freshly prepared, or produced from the water infusing powder, or the water-soluble powder) comprise one or more additional juices. Non-limiting examples of juices include cranberry juice, orange juice, grapefruit juice, aloe vera juice, apple juice, beet juice, cantaloupe juice, carrot juice, celery juice, cherry juice, lime juice, clam juice, coconut water, cucumber juice, dandelion-green juice, Durian milk, grape juice, guava juice, honeydew juice, kaffir lime juice, kiwifruit juice, lemonade, limeade, limonana, lychee juice, melon juice, papaya juice, parsley juice, pineapple juice, pog (passion fruit-orange-guava), pomegranate juice, prune juice, raspberry vinegar, salgam, carrot juice, strawberry juice, spinach juice, sugarcane juice, tejuino, tomato juice, turnip juice, vegetable juice, watercress juice, wheatgrass juice, and winter melon punch. In some embodiments, the Cannabis (hemp) and coffee fruit juice compositions of the present disclosure (whether freshly prepared, or produced from the water infusing powder, or the water-soluble powder) comprise a soda drink. Non-limiting examples of sodas include soda water, club soda, tonic water, ginger ale, ginger beer, lemon-lime soda, cola, and root beer. In some embodiments, sodas may be selected from the group consisting of Coca-Cola®, Caffeine Free Coca-Cola®, Coca-Cola® Black cherry vanilla, Coca-Cola® C2, Coca-Cola® Clear, Coca-Cola® Citra, Coca-Cola® Light, Coca-Cola® Orange, Coca-Cola® Orange Vanilla, Coca-Cola® Raspberry, Coca-Cola® Vanilla, Coca-Cola® with lemon, Coca-Cola® with lime, Coca-Cola® Zero, Diet Coke®, Diet Coke® with lemon, Diet Coke® lime, Diet Coke® plus, Diet Coke® with zesty blood orange, Diet Coke® with citrus zest, Fresca®, Mr. Pibb®, Sprite®, Sprite Cranberry®, Sprite Ice®, Sprite Zero®, Mountain Dew®, Pepsi®, Diet Pepsi®, Pepsi® Cola, Pepsi® Lime, Sierra Mist®, Keurig Dr Pepper®, Dr Pepper®, A&W Root Bear®, A&W Cream Soda®, and 7 Up®. Non-limiting examples of milks include whole milk, reduced fat milk, 2% milk, low fat milk, skim milk, organic milk, lactose free milk, flavored milk, raw milk, almond milk, soy milk, rice milk, hemp milk, flax milk, hazelnut milk, coconut milk, oat milk, cashew milk, and goat milk.

Flavorings

In some embodiments, the compositions of the present disclosure (i.e., the powdered compositions, or the beverages) comprise flavorings. Non-limited examples of flavorings include vanilla, chocolate, hazelnut, caramel, cinnamon, mint, eggnog, apple, apricot, aromatic bitters, banana, berry, blackberry, blueberry, celery, cherry, cranberry, strawberry, raspberry, juniper berry, brandy, cachaca, carrot, citrus, lemon, lime, orange, grapefruit, tangerine, coconut, cola, menthol, gin, ginger, licorice, hot, milk, nut, almond, macadamia nut, peanut, pecan, pistachio, walnut, peach, pear, pepper, pineapple, plum, quinine, rum, white rum, dark rum, sangria, shellfish, clam, tea, black tea, green tea, tequila, tomato, top note, tropical, vermouth, dry vermouth, sweet vermouth, whiskey, bourbon whiskey, Irish whiskey, rye whiskey, Scotch whisky, Canadian whiskey, red pepper, black pepper, horseradish, wasabi, jalapeno pepper, chipotle pepper essential oils, concretes, absolutes, resins, resinoids, balms, tinctures, soybean oil, coconut oil, palm oil, kern oil, sunflower oil, peanut oil, almond oil, cocoa butter, amyris oil, angelica seed oil, angelica root oil, aniseed oil, anise seed oil, valerian oil, basil oil, tarragon oil, eucalyptus citriodora oil, eucalyptus oil, fennel oil, fir needle oil, galbanum oil, galbanum resin, geranium oil, grapefruit oil, guaiac wood oil, guaiac balsam, guaiac balsam oil, helichrysum absolute, helichrysum oil, ginger oil, iris root absolute, iris root oil, jasmin absolute, calmus oil, chamomile oil bleu, chamomile oil roman, carrot seed oil, cascarilla oil, mint oil, carvi oil, labdanum oil, labdanum absolute, labdanum resin, lavandin absolute, lavandin oil, lavender absolute, lavender oil, lemongrass oil, Bursera penicillata (linaloe) oil, litseacubeba oil, bay laurel leaf oil, macis oil, marjoram oil, mandarin oil, massoirinde oil, mimosa absolute, ambrette seed oil, ambrette tincture, muskatelle salbei oil, nutmeg oil, orange blossom absolute, orange oil, orange pulp, dehydrated orange pulp powder, orange zest, oregano oil, palmarosa oil, patchouli oil, perilla oil, parsley leaf oil, parsley seed oil, clove seed oil, peppermint oil, pepper oil, pimento oil, pine oil, poley oil, rose absolute, rose wood oil, rose oil, rosemary oil, sage oil, lavandin, sage oil Spanish, sandalwood oil, celery seed oil, lavender spike oil, star anis oil, styrax oil, tagetes oil, pine needle oil, tea-tree oil, turpentine oil, thyme oil, tolu balm, tonka absolute, tuberose absolute, vanilla extract, violet leaf absolute, verbena oil, vetiver oil, juniper berry oil, wine yeast oil, wormwood oil, wintergreen oil, ylang oil, hyssop oil, civet absolute, cinnamon leaf oil, cinnamon bark oil or any other type of food flavoring or edible substance or a combination thereof.

Sweeteners

In some embodiments, the compositions of the present disclosure (i.e., the powdered compositions, or the beverages) comprise a sweetener. In some embodiments, the sweetener is sugar. In some embodiments, the sweetener is granulated sugar. In some embodiments, one or more sweeteners are selected from a group consisting of sugar, granulated sugar, stevia, sucralose, erythritol, xylitol, yacon syrup, cane sugar, fructose, corn syrup, crystalline fructose, dextrose, malto-dextrose, maltodextrin, glycerine, threitol, erythritol, rebaudioside A, stevia, xylitol, arabitol, ribitol, sorbitol, mannitol, maltitol, maltotriitol, maltotetraitol, lactitol, hydrogenated isomaltulose, hydrogented starch, shellac, ethyl cellulose, hydroxy propyl methylcellulose, starches, modified starches, carboxyl cellulose, carrageenan, cellulose acetate phthalate, cellulose acetate trimellitate, chitosan, corn syrup solids, dextrins, fatty alcohols, hydroxy cellulose, hydroxy ethyl cellulose, hydroxy methyl cellulose, hydroxy propyl cellulose, hydroxy propyl ethyl cellulose, hydroxy propyl methyl cellulose, hydroxy propyl methyl cellulose phthalate, polyethylene glycol or a combination thereof. In addition, the sweetener may have various levels of granularity. For example, granulated sugar, baker's sugar, sanding sugar, etc. may be used. In some embodiments a more highly granulated sweetener is used.

Stabilizers

In some embodiments, the compositions of the present disclosure (i.e., the powdered compositions, or the beverages) comprise a stabilizer. In embodiments, the introduction of a stabilizer increases the shelf life of the product. In embodiments, dried powders and/or beverages or the present disclosure are stable for up to one year or up to two years. In embodiments, a gum-based stabilizer is utilized. Non-limiting examples of gum-based stabilizers include, pectin, alginate, carboxymethyl cellulose, locust bean gum, xanthan gum, gellan gum, guar gum, carrageenan, gum ghatti, karaya gum, tragacanth, gum arabic, gelatin and mixtures thereof.

In some embodiments, the compositions of the present disclosure (i.e., the powdered compositions, or the beverages) comprise one or more added minerals. Non-limiting examples of minerals that can be included in the compositions of the present invention include calcium, magnesium, zinc, iodine, and copper. Any soluble salt of these minerals suitable for inclusion edible products can be used, for example, calcium carbonate, calcium citrate, calcium malate, calcium-citrate-malate, calcium gluconate, magnesium citrate, magnesium gluconate, magnesium sulfate, zinc chloride, zinc sulfate, potassium iodide, and copper sulfate.

Anti-Microbials

In some embodiments, the compositions of the present disclosure (i.e., the powdered compositions, or the beverages) comprise one or more anti-microbials. Food products, such as fruit powders and juice beverages, when exposed to food spoilage microorganisms, can provide a hospitable environment for rapid microbial growth. Such exposure can result from accidental inoculation of the dilute juice beverage with these microorganisms during manufacturing, packaging, or consumer handling. Food spoilage microorganisms can then rapidly proliferate by feeding on nutrients provided by the juice component of the dilute juice beverage. In embodiments, preservatives, such as sorbates, benzoates, organic acids, and combinations thereof are added to food products to provide some degree of microbial inhibition.

Thickeners

In some embodiments, the compositions of the present disclosure (i.e., the powdered compositions, or the beverages) comprise thickeners. Thickeners enable regulation of the consistency of beverages. Non-limiting examples of thickeners include xanthan gum, carboxymethylcellulose, propylene glycol alginate, gellan gum, guar gum, pectin, tragacanth gum, gum acacia, locust bean gum, gum arabic, gelatin, as well as mixtures of these thickeners.

In some embodiments, the juice beverages of the present invention comprise an emulsion. In some embodiments the emulsion is a cloud emulsion or a flavor emulsion. In some embodiments, for cloud emulsions, the clouding agent can comprise one or more fats or oils stabilized as an oil-in-water emulsion using a suitable food grade emulsifier. In some embodiments, any of a variety of fats or oils can be employed as the clouding agent, provided that the fat or oil is suitable for use in foods and beverages. In some embodiments the fat or oil is refined, bleached and deodorized to remove off-flavors. In some embodiments, clouding agents are those fats that are organoleptically neutral, including vegetable fats such as soybean, corn, safflower, sunflower, cottonseed, canola, and rapeseed; nut fats such as coconut, palm, and palm kernel; and synthetic fats. See U.S. Pat. No. 4,705,691 (Kupper, et al), issued Nov. 10, 1987 (herein incorporated by reference) for suitable fat or oil clouding agents. In embodiments, any suitable food grade emulsifier can be used that can stabilize the fat or oil clouding agent as an oil-in-water emulsion. Suitable emulsifiers include gum acacia, modified food starches (e.g., alkenylsuccinate modified food starches), anionic polymers derived from cellulose (e.g., carboxymethylcellulose), gum ghatti, modified gum ghatti, xanthan gum, tragacanth gum, guar gum, locust bean gum, pectin, and mixtures thereof. See U.S. Pat. No. 4,705,691 (Kupper, et al), issued Nov. 10, 1987, which is incorporated by reference.

In some embodiments, the present disclosure teaches emulsifiers, including modified starches treated so they contain hydrophobic as well as hydrophilic groups, such as those described in U.S. Pat. No. 2,661,349 (Caldwell et al) (herein incorporated by reference). In some embodiments, the emulsifier of the present disclosure includes octenyl succinate (OCS) modified starches such as those described in U.S. Pat. No. 3,455,838 (Marotta et al.), and U.S. Pat. No. 4,460,617 (Barndt et al.), (herein incorporated by reference).

In some embodiments, the clouding agent can be combined with a weighting agent to provide a beverage opacifier that imparts a total or partial opaque effect to the beverage without separating out and rising to the top. In some embodiments, the beverage opacifier provides the appearance to the consumer of a juice-containing beverage. In some embodiments, any suitable weighting oil can be employed in the beverage opacifier. A non-limiting list of weighting oils include brominated vegetable oil, glycerol ester of wood rosin (ester gum), sucrose acetate isobutyrate (SAIB) and other sucrose esters, gum damar, colophony, gum elemi, or others known to those skilled in the art. Other suitable weighting agents include brominated liquid polyol polyesters that are nondigestible. See U.S. Pat. No. 4,705,690 (Brand et al), issued Nov. 10, 1987, which is incorporated by reference. In some embodiments, the cloud/opacifier emulsion is prepared by mixing the clouding agent with the weighting agent (for opacifier emulsions), the emulsifier and water. In some embodiments, the emulsion typically contains from about 0.1 to about 25% clouding agent, from about 1 to about 20% weighting oil agent (in the case of opacifier emulsions), from about 1 to about 30% emulsifiers, and from about 25 to about 97.9% water, quantum satis.

Other Enriching Agents

In some embodiments, the compositions of the present disclosure can undergo an optional enrichment step. Non-limiting examples of enriching substances are coffee fruit extract, concentrated coffee, dried coffee, coffee oils, coffee aromas (distillates), flavor powders, flavor oils, spices, ground or pulverized cocoa beans, ground or pulverized vanilla beans, vitamins, antioxidants, nutraceuticals, dietary fiber, an omega-3 oil, an omega-6 oil, an omega-9 oil, a flavonoid, lycopene, selenium, a beta-carotene, resveratrol, a vegetable extract, dry green coffee extract, wet green coffee extract and an herbal extract.

Nutrients

In some embodiments, the compositions of the present disclosure are enriched with one or more nutrients. Non-limiting examples of nutrients include fiber, protein, carbohydrates, antioxidants, iodized salt, folate, niacin, vitamin D, whey protein, soy protein, vegetable protein, vitamin A, riboflavin, thiamin, vitamin A, vitamin B6, vitamin B12, vitamin D, vitamin E, calcium, fluoride, iodine, iron, selenium, and zinc.

Additional Formulations of the Present Disclosure

In some embodiments, the present disclosure teaches additional formulations for the disclosed powders. In some embodiments, the present disclosure teaches tablets or capsules comprising the water infusing or water-dissolvable powders of the present disclosure. That is, in some embodiments, the present disclosure teaches tablets or capsules comprising dehydrated Cannabis (hemp) juice or pulp, and/or dehydrated coffee fruit juice or pulp, and/or dehydrated orange juice pulp. In some embodiments, the present disclosure teaches tablets or capsules comprising the dehydrated orange pulp powder.

Persons with skill in the art will be familiar with techniques for forming tablets or capsules from powders. In some embodiments, the tablets of the present disclosure are produced via one or a combination of methods selected from the group consisting of: (1) dry mixing, (2) direct compression, (3) milling, (4) dry or non-aqueous granulation, (5) melt granulation, or (6) fusion. See, Lachman et al., The Theory and Practice of Industrial Pharmacy (1986).

In some embodiments, the present disclosure teaches compressed tablets, which are prepared by compacting a formulation containing an acid-labile pharmaceutical agent and/or buffering agent and/or excipient selected to aid the processing and improve the properties of the product. The term “compressed tablet” generally refers to a plain, uncoated tablet for oral ingestion, prepared by a single compression or by pre-compaction tapping followed by a final compression.

In some embodiments, the tablets or pills of the present invention may be coated or otherwise compounded to provide a dosage form affording the advantage of improved handling or storage characteristics. For example, the tablet or pill can comprise an inner dosage and an outer dosage component, the latter being in the form of an envelope over the former.

In some embodiments, the tablets of the present disclosure are produced via wet granulation. Wet granulation is a process of using a liquid binder to lightly agglomerate a powder mixture. In some embodiments, wet granulation follows the following steps: first, the active ingredients (e.g., comprising the Cannabis (hemp) juice powder and coffee fruit juice powder), and excipients are weighed and mixed. Next, the wet granulate is prepared by adding the liquid binder-adhesive to the powder blend and mixing thoroughly. Examples of binders/adhesives include aqueous preparations of cornstarch, natural gums such as acacia, cellulose derivatives such as methyl cellulose, gelatin, and povidone. After mixing, the resulting paste is screened through a mesh to form pellets or granules. The pellets or granules are dried using e.g., a conventional tray-dryer or fluid-bed dryer.

In some embodiments, the tablets of the present disclosure are produced via dry granulation. Dry granulation processes create granules by light compaction of the powder blend under low pressures. The compacts so-formed are broken up gently to produce granules (agglomerates). This process is often used when the product to be granulated is sensitive to moisture and heat. Dry granulation can be conducted on a tablet press using slugging tooling or on a roll press called a roller compactor. Dry granulation equipment offers a wide range of pressures to attain proper densification and granule formation. Dry granulation requires drugs or excipients with cohesive properties, and a “dry binder” may need to be added to the formulation to facilitate the formation of granules.

Exemplary binders include celluloses such as hydroxypropylcellulose, methylcellulose, and hydroxypropylmethylcellulose; starches such as corn starch, pregelatinized starch, and hydroxpropyl starch; sugars such as glucose, dextrose, sucrose, lactose and sorbitol; alcohols such as polyvinyl alcohol and polyethylene glycol; waxes and natural and synthetic gums such as acacia, tragacanth, sodium alginate; synthetic polymers such as polymethacrylates and polyvinylpyrrolidone; and povidone, dextrin, pullulane, agar, gelatin, tragacanth, macrogol, or combinations thereof.

In some embodiments, the tablets of the present disclosure comprise a coating. Exemplary coating materials include hydroxypropylmethyl cellulose 2910, aminoalkyl methacrylate copolymer E, polyvinylacetal diethylaminoacetate, macrogol 6000, titanium oxide, or combinations thereof. Exemplary plasticizers include triethyl citrate, triacetin, macrogol 6000, or combinations thereof.

In some embodiments, the powders of the present disclosure are formulated into capsules. Capsule forms compatible with the powders of the present disclosure are published in U.S. Pat. Nos. 4,800,083; 4,532,126; 4,935,243; 6,258,380; each of which is incorporated herein by reference in its entirety.

It is to be understood that the description above as well as the examples that follow are intended to illustrate, and not limit, the scope of the invention. Other aspects, advantages and modifications within the scope of the invention will be apparent to those skilled in the art to which the invention pertains.

### EXAMPLES

### Example 1. Preparation of Insoluble Coffee Fruit/Hemp Fruit Powder (Water Infusing Powder)

Mature unpollinated female inflorescences from Cannabis sativa (hemp) plants were harvested by hand and placed in bins for transport to processing facility. Fresh (non-dried) inflorescences were ground in a wet mill grinder to produce fresh hemp pulp. Coffee fruit was harvested from coffee plants at maturity (when fruit exhibits a deep red or purple color) and placed in a bin for transport. The freshly harvested coffee fruit was ground in a MDP60 Disc Mill wet mill grinder (Penagos), with the complete ground fruit collected (for use in whole-fruit products). In some batches, the freshly harvested fruit was first de-beaned in the machine, to separate the coffee fruit (mesocarp) from the coffee bean. The coffee fruit mesocarp was collected from the wet mill in the form of a de-beaned coffee fruit pulp. The coffee fruit pulp (with or without the bean) was added to a mixing tank with the hemp pulp, ascorbic acid, and water, and mixed for about two minutes. The mixture of coffee fruit pulp, hemp pulp, ascorbic acid, and water was dehydrated using an ultrasound pulverization system. The ultrasound pulverization system removed water from the mixture, thereby producing a water insoluble, dried powder from the coffee fruit pulp and hemp pulp. The water-insoluble powder was placed in a microwave dehydrator to remove any additional moisture and kill off any mycotoxins or mold.

### Example 2. Preparation of Insoluble Hemp Fruit/Orange Pulp Powder (Water Infusing Powder)

Mature unpollinated female inflorescences from Cannabis sativa (hemp) plants will be harvested by hand and placed in bins for transport to processing facility. Fresh (non-dried) inflorescences will be ground in a wet mill grinder to produce fresh hemp pulp. Left over orange peels and pulp will be recovered from oranges used for orange juice production (“juiced oranges”). The juiced oranges will then be ground in a wet mill to produce an orange pulp. The hemp pulp, and orange pulp will be added to a mixing tank and mixed for about two minutes. The mixture of hemp pulp and orange pulp will be dehydrated using an ultrasound pulverization system. The ultrasound pulverization system removed water from the mixture, thereby producing a water insoluble, dried powder. The water-insoluble powder will be placed in a microwave dehydrator to remove any additional moisture and kill off any mycotoxins or mold.

### Example 3. Preparation of Insoluble Coffee Fruit/Hemp Fruit/Orange Pulp Powder (Water Infusing Powder)

Mature unpollinated female inflorescences from Cannabis sativa (hemp) plants will be harvested by hand and placed in bins for transport to processing facility. Fresh (non-dried) inflorescences will be ground in a wet mill grinder to produce fresh hemp pulp. Coffee fruit will be harvested from coffee plants at maturity (when fruit exhibits a deep red color or purple color) and placed in a bin for transport. The freshly harvested coffee fruit will be ground in a wet mill grinder, with the complete ground fruit collected (for use in whole-fruit products). In some batches, the freshly harvested fruit will be first de-beaned in the machine, to separate the coffee fruit (mesocarp) from the coffee bean. The coffee fruit mesocarp will be collected from the wet mill in the form of a de-beaned coffee fruit pulp. Left over orange peels and pulp will be recovered from oranges used for orange juice production (“juiced oranges”). The juiced oranges will be ground in a wet mill to produce an orange pulp. The coffee fruit pulp (with or without the bean) will be added to a mixing tank with the hemp pulp, and orange pulp, and mixed for about two minutes. In some batches, ascorbic acid will be added before mixing. The mixture of coffee fruit pulp, hemp pulp, orange pulp, and ascorbic acid will be dehydrated using an ultrasound pulverization system. The ultrasound pulverization system removed water from the mixture, thereby producing a water insoluble, dried powder from the coffee fruit pulp, hemp pulp, and orange pulp. The water-insoluble powder will be placed in a microwave dehydrator to remove any additional moisture and kill off any mycotoxins or mold.

### Example 4. Preparation of Water-Soluble Coffee Fruit/Hemp Fruit Powder

Hemp juice and coffee fruit juice were utilized to prepare a water-soluble coffee fruit/hemp fruit powder. Industrial hemp was harvested from Cannabis sativa as described in Example 1. The leafy upper part of the hemp, including the flower heads, were cold pressed in a cold press to form hemp juice. The coffee fruit juice was prepared by harvesting coffee fruit from a mature coffee plant. Coffee fruit was placed in an extruder press and crushed to form a coffee fruit juice with a sugar content of about 3° Bx to 4° Bx. The coffee fruit juice was fortified with coffee fruit extract. Coffee fruit juice was combined with hemp juice in a 3:1 mass ratio. The mixture of coffee fruit juice and hemp juice was spray-dried to produce a dried powder. The resultant water-soluble powder was placed into a grinder to further break up the powder.

### Example 5. Preparation of Water-Soluble Orange Fruit/Hemp Fruit Powder

Hemp juice and orange fruit juice will be utilized to prepare a water-soluble coffee fruit/hemp fruit powder. Industrial hemp will be harvested from Cannabis sativa as described in Example 1. The leafy upper part of the hemp, including the flower heads, will be cold pressed in a cold press to form hemp juice. Commercially purchased orange juice will be combined with hemp juice in a 3:1 mass ratio. The mixture of orange fruit juice and hemp juice will be spray-dried to produce a dried powder. The resultant water-soluble powder will be placed into a grinder to further break up the powder.

### Example 6. Preparation of Water-Soluble Coffee Fruit/Orange Fruit/Hemp Fruit Powder

Hemp juice, orange juice, and coffee fruit juice will be utilized to prepare a water-soluble powder. Industrial hemp will be harvested from Cannabis sativa as described in Example 1. The leafy upper part of the hemp, including the flower heads, will be cold pressed in a cold press to form hemp juice. The coffee fruit juice will be prepared by harvesting coffee fruit from a mature coffee plant. Coffee fruit will be placed in an extruder press and crushed to form a coffee fruit juice with a sugar content of about 3° Bx to 4° Bx. The coffee fruit juice will be fortified with coffee fruit extract and dehydrated orange pulp powder. Commercially purchased orange juice will be combined with the produced coffee fruit juice and hemp juice in a 1:1:1 mass ratio. The mixture of orange juice, coffee fruit juice, and hemp juice will be spray-dried to produce a powder. The resultant water-soluble powder will be placed into a grinder to further break up the powder.

### Example 7. Preparation of Beverage from Water-Insoluble (Water-Infusing) Hemp and Coffee Fruit Powder

The water-insoluble powder of Example 1 was placed in a filter bag. The filter bag containing the water-insoluble powder was steeped in warm water for five minutes, and then removed. The remaining water had been infused with the flavors of the water-insoluble powder to produce a tea-like drink.

### Example 8. Preparation of Beverage Comprising Water-Insoluble (Water-Infusing) Hemp and Orange Pulp Powder

The water-insoluble powder of Example 2 will be placed in a filter bag. The filter bag containing the water-insoluble powder will be steeped in warm water for five minutes, and then removed. The remaining water will be infused with the flavors of the water-insoluble powder to produce a tea-like drink.

### Example 9. Preparation of Beverage Comprising Water-Insoluble (Water-Infusing) Hemp and Coffee Fruit Powder and Orange Pulp Powder

The water-insoluble powder of Example 3 will be placed in a filter bag. The filter bag containing the water-insoluble powder will be steeped in warm water for five minutes, and then removed. The remaining water will be infused with the flavors of the water-insoluble powder to produce a tea-like drink.

### Example 10. Preparation of Beverage Comprising Water-Soluble Hemp Fruit Juice and Coffeefruit Juice Powder

The water-soluble powder of Example 4 was mixed with cold water to form a juice-like drink.

### Example 11. Preparation of Beverage Comprising Water-Soluble Hemp Fruit Juice, and Orange Juice Powder

The water-soluble powder of Example 5 will be mixed with cold water to form a juice-like drink.

### Example 12. Preparation of Beverage Comprising Water-Soluble Hemp Fruit Juice, Coffee Fruit Juice, and Orange Juice Powder

The water-soluble powder of Example 6 will be mixed with cold water to form a juice-like drink.

### Example 13. Preparation of Lemon-Ginger Hemp Fruit Juice, Coffee Fruit Juice Beverage

One inch of fresh ginger root was grated and added to a cup, followed by one tablespoon of lemon juice. The water-soluble powder of Example 4 was added to the receptacle together with some water to produce a lemon-ginger juice with coffee fruit and hemp juice.

### Example 14. Preparation of Lemon-Ginger-Orange Hemp Fruit Juice, Coffee Fruit Juice Beverage

One inch of fresh ginger root will be grated and added to a cup, followed by one tablespoon of lemon juice. The water-soluble powder of Example 5 will be added to the receptacle together with some water to produce a lemon-ginger-orange juice with coffee fruit and hemp juice.

### Example 15. Preparation of Lemon-Ginger Hemp Fruit, Coffee Fruit Tea Beverage

One inch of fresh ginger root was grated and added to a cup, followed by one tablespoon of lemon juice. The water-insoluble powder was added to a filter bag as in Example 7 and placed in the cup containing lemon juice and grated ginger with warm water. The filter bag was steeped in the warm water for about five minutes before being removed. The remaining water was a tea-like drink.

### Example 16. Preparation of Lemon-Ginger-Orange Hemp Fruit, Coffee Fruit Tea Beverage

One inch of fresh ginger root will be grated and added to a cup, followed by one tablespoon of lemon juice. The water-insoluble powder will be added to a filter bag as in Example 9 and placed in the cup containing lemon juice and grated ginger with warm water. The filter bag will be steeped in warm water for about five minutes before being removed. The resulting water will be a tea-like drink.

### Example 17. Preparation of Hemp Juice, Coffee Fruit Juice Beverage

Hemp juice and coffee fruit juice produced using a cold press (as described in Example 4) were added to a cup in a 1:3 (w/w) ratio to produce a juice-like drink.

### Example 18. Preparation of an Orange, Hemp Juice, Coffee Fruit Juice Beverage

Hemp juice and coffee fruit juice will be produced using a cold press (as described in Example 6) and commercially purchased orange juice will be added to a cup in a 1 (orange):1 (coffee fruit):1 (hemp) (w/w) ratio to produce a juice-like drink.

### Example 19. Formulations of Coffee Fruit/Hemp Fruit Containing Beverages

Several different beverages comprising varying ratios (w/w) of hemp pulp/juice to coffee fruit pulp/juice can be prepared, including 1:1 w/w, 1:2 w/w, 1:3 w/w, 1:4 w/w, 1:5 w/w, 5:1 w/w, 4:1 w/w, 3:1 w/w, and 2:1 w/w. Table 1 provides a summary of the possible formulations.

TABLE 1
Formulations of coffee fruit/hemp fruit containing beverages

| | Coffee Fruit | Hemp Inflorescence |
|---|---|---|
| | (% fresh mass weight) | (% fresh mass weight) |
| Water- | 20% of pulp | 80% of pulp |
| infusible | 30% of pulp | 70% of pulp |
| powder | 40% of pulp | 60% of pulp |
| (1 g steeped in | 50% of pulp | 50% of pulp |
| 1L of water) | 60% of pulp | 40% of pulp |
| | 70% of pulp | 30% of pulp |
| | 80% of pulp | 20% of pulp |
| | 90% of pulp | 10% of pulp |
| | 100% of pulp | 0% of pulp |
| Water-soluble | 0% of juice | 100% of juice |
| powder | 10% of juice | 90% of juice |
| (1 g dissolved in | 20% of juice | 80% of juice |
| 1 L of water) | 30% of juice | 70% of juice |
| | 40% of juice | 60% of juice |
| | 50% of juice | 50% of juice |
| | 60% of juice | 40% of juice |
| | 70% of juice | 30% of juice |
| | 80% of juice | 20% of juice |
| | 90% of juice | 10% of juice |
| | 100% of juice | 0% of juice |
| Fresh Juice | 0% of juice | 100% of juice |
| | 10% of juice | 90% of juice |
| | 20% of juice | 80% of juice |
| | 30% of juice | 70% of juice |
| | 40% of juice | 60% of juice |
| | 50% of juice | 50% of juice |
| | 60% of juice | 40% of juice |
| | 70% of juice | 30% of juice |
| | 80% of juice | 20% of juice |
| | 90% of juice | 10% of juice |
| | 100% of juice | 0% of juice |

### Example 20. Formulations of Coffee Fruit/Hemp Fruit/Orange Containing Beverages

Several different beverages comprising orange pulp/juice, hemp pulp/juice, and coffee fruit pulp/juice can be prepared. Orange pulp/juice, hemp pulp/juice, and coffee fruit pulp/juice can be mixed in varying weight ratios, including 0:0:5 w/w, 0:1:4 w/w, 0:2:3 w/w, 0:3:2 w/w, 0:4:1 w/w, 0:5:0 w/w, 1:0:4 w/w, 1:1:3 w/w, 1:2:2 w/w, 1:3:1 w/w, 1:4:0 w/w, 2:0:3 w/w, 2:1:2 w/w/w, 2:2:1 w/w, 3:0:2 w/w, 3:1:1 w/w, 3:2:0 w/w, 4:0:1 w/w, 4:1:0 w/w, and 5:0:0 w/w. Table 2 provides a summary of the possible formulations.

TABLE 2
Formulations of coffee fruit/hemp fruit/orange containing beverages

| | Coffee Fruit | | Orange |
|---|---|---|---|
| | (% fresh | Hemp Inflorescence | (% fresh |
| | mass weight) | (% fresh mass weight) | mass weight) |
| Water- | 0% of pulp | 0% of pulp | 100% of pulp |
| infusible | 0% of pulp | 20% of pulp | 80% of pulp |
| powder | 0% of pulp | 40% of pulp | 60% of pulp |
| (1 g | 0% of pulp | 60% of pulp | 40% of pulp |
| steeped in | 0% of pulp | 80% of pulp | 20% of pulp |
| 1 L of | 0% of pulp | 100% of pulp | 0% of pulp |
| water) | 20% of pulp | 0% of pulp | 80% of pulp |
| | 20% of pulp | 20% of pulp | 60% of pulp |
| | 20% of pulp | 40% of pulp | 40% of pulp |
| | 20% of pulp | 60% of pulp | 20% of pulp |
| | 20% of pulp | 80% of pulp | 0% of pulp |
| | 40% of pulp | 0% of pulp | 60% of pulp |
| | 40% of pulp | 20% of pulp | 40% of pulp |
| | 40% of pulp | 40% of pulp | 20% of pulp |
| | 60% of pulp | 0% of pulp | 40% of pulp |
| | 60% of pulp | 20% of pulp | 20% of pulp |
| | 60% of pulp | 40% of pulp | 0% of pulp |
| | 80% of pulp | 0% of pulp | 20% of pulp |
| | 80% of pulp | 20% of pulp | 0% of pulp |
| | 100% of pulp | 0% of pulp | 0% of pulp |
| Water- | 0% of juice | 0% of juice | 100% of juice |
| soluble | 0% of juice | 20% of juice | 80% of juice |
| powder | 0% of juice | 40% of juice | 60% of juice |
| (1g | 0% of juice | 60% of juice | 40% of juice |
| dissolved | 0% of juice | 80% of juice | 20% of juice |
| in 1 L of | 0% of juice | 100% of juice | 0% of juice |
| water) | 20% of juice | 0% of juice | 80% of juice |
| | 20% of juice | 20% of juice | 60% of juice |
| | 20% of juice | 40% of juice | 40% of juice |
| | 20% of juice | 60% of juice | 20% of juice |
| | 20% of juice | 80% of juice | 0% of juice |
| | 40% of juice | 0% of juice | 60% of juice |
| | 40% of juice | 20% of juice | 40% of juice |
| | 40% of juice | 40% of juice | 20% of juice |
| | 60% of juice | 0% of juice | 40% of juice |
| | 60% of juice | 20% of juice | 20% of juice |
| | 60% of juice | 40% of juice | 0% of juice |
| | 80% of juice | 0% of juice | 20% of juice |
| | 80% of juice | 20% of juice | 0% of juice |
| | 100% of juice | 0% of juice | 0% of juice |
| Fresh | 0% of juice | 0% of juice | 100% of juice |
| Juice | 0% of juice | 0% of juice | 100% of juice |
| | 0% of juice | 20% of juice | 80% of juice |
| | 0% of juice | 40% of juice | 60% of juice |
| | 0% of juice | 60% of juice | 40% of juice |
| | 0% of juice | 80% of juice | 20% of juice |
| | 0% of juice | 100% of juice | 0% of juice |
| | 20% of juice | 0% of juice | 80% of juice |
| | 20% of juice | 20% of juice | 60% of juice |
| | 20% of juice | 40% of juice | 40% of juice |
| | 20% of juice | 60% of juice | 20% of juice |
| | 20% of juice | 80% of juice | 0% of juice |
| | 40% of juice | 0% of juice | 60% of juice |
| | 40% of juice | 20% of juice | 40% of juice |
| | 40% of juice | 40% of juice | 20% of juice |
| | 60% of juice | 0% of juice | 40% of juice |
| | 60% of juice | 20% of juice | 20% of juice |
| | 60% of juice | 40% of juice | 0% of juice |
| | 80% of juice | 0% of juice | 20% of juice |
| | 80% of juice | 20% of juice | 0% of juice |
| | 100% of juice | 0% of juice | 0% of juice |
| | 0% of juice | 0% of juice | 100% of juice |

### Example 21. Preparation of Dehydrated Orange Pulp Powder

Left over orange peels and pulp was recovered from oranges used for orange juice production (“juiced oranges”). The juiced oranges were then ground in a wet mill to produce an orange pulp. The orange pulp was dehydrated using an ultrasound pulverization system. The ultrasound pulverization system removed water from the mixture and further broke up the orange tissue, thereby producing a dehydrated orange pulp powder. The dehydrated orange pulp powder was placed in a microwave dehydrator to remove any additional moisture and kill off any mycotoxins or mold.

### Example 22. Preparation of Tablet Containing Water-Soluble Coffee Fruit/Orange Fruit/Hemp Fruit Powder

The powders obtained in Examples 1-6 will be blended with excipients and directly compressed into tablets for oral administration. The components used for tablet preparation are given in Table 3.

TABLE 3
Composition of Tablet comprising the powders of Examples 1-6

| Component | Weight % |
|---|---|
| A powder of Examples 1-6 | 75 |
| Lactose | 23.8 |
| Magnesium stearate | 1 |

### Example 23. Preparation of a Formulation of a Beverage Containing Water-Infusing Hemp and Coffee Fruit Powder

Cannabis sativa (hemp) plants were harvested by hand and placed in bins for transport to processing facility. The leafy upper part of the fresh (non-dried) hemp, including stems, was pulverized to produce a wet hemp blend. Water-infusing coffee fruit powder was prepared as described in Example 1.

To prepare 12 oz beverage containing water-infusing hemp and coffee fruit powder, 0.1-50 grams (preferably 2-10 grams) coffee fruit powder was added to water, mixed with 0.1-300 grams (preferably 10-50 grams) of the wet hemp blend and cold brewed for 12-24 hours. The mixture can be stored in a fridge or stored under similar conditions. The mixture was then strained and squeezed to produce a beverage base.

Another method to prepare 12 oz beverage containing water-infusing hemp and coffee fruit powder is adding 0.1-50 grams (preferably 2-10 grams) coffee fruit powder to water and bring to boil. The coffee fruit powder was then steeped in the boiled water for twenty minutes to produce a tea-like drink. 0.1-300 grams (preferably 10-50 grams) of the wet hemp blend was added to water, strained and squeezed to produce a liquid. The liquid and the tea-like coffee drink were then mixed to produce a beverage base.

Another method to prepare 12 oz beverage containing water-infusing hemp and coffee fruit powder is adding 0.1-50 grams (preferably 2-10 grams) coffee fruit powder to water and mix with 0.1-300 grams (preferably 10-50 grams) of the wet hemp blend.

### Example 24. Preparation of a Formulation of a Beverage Containing Water-Infusing Hemp and Orange Pulp Powder

Wet hemp blend was prepared as described in Example 23. Water-infusing orange pulp powder was prepared as described in Example 2.

To prepare 12 oz beverage containing water-infusing hemp and orange pulp powder, 0.1-300 grams (preferably 10-50 grams) of the wet hemp blend was added to water, strained and squeezed to produce a liquid. 0.1-50 grams (preferably 2-10 grams) orange pulp powder was added to water and cold brewed for 12-24 hours in a fridge or stored under similar conditions to create a tea-like drink. The hemp liquid was then mixed with the tea-like drink to produce a beverage base.

Another method to prepare 12 oz beverage water-infusing hemp and orange pulp powder is adding 0.1-50 grams (preferably 2-10 grams) orange pulp powder to water and bring to boil. The orange pulp powder was then steeped in the boiled water for twenty minutes to produce a tea-like drink. 0.1-300 grams (preferably 10-50 grams) of the wet hemp blend was added to water, strained and squeezed to produce a liquid. The liquid and the tea-like drink were then mixed to produce a beverage base.

### Example 25. Preparation of a Formulation of a Beverage Containing Water-Infusing Hemp, Coffee Fruit, and Orange Pulp

Wet hemp blend was prepared as described in Example 23. Water-infusing coffee fruit powder was prepared as described in Example 1. Water-infusing orange pulp powder was prepared as described in Example 2.

To prepare 12 oz beverage containing water-infusing hemp, coffee fruit, and orange pulp, 0.1-50 grams (preferably 2-10 grams) coffee fruit powder and 0.1-50 grams (preferably 2-10 grams) orange fruit powder were added to water, mixed with 0.1-300 grams (preferably 10-50 grams) of the wet hemp blend and cold brewed for 12-24 hours. The mixture can be stored in a fridge or stored under similar conditions. The mixture was then strained and squeezed to produce a beverage base.

Another method to prepare 12 oz beverage containing water-infusing hemp, coffee fruit, and orange pulp is adding 0.1-50 grams (preferably 2-10 grams) coffee fruit powder and 0.1-50 grams (preferably 2-10 grams) orange pulp powder to water and bring to boil. The coffee fruit powder and orange pulp powder were then steeped in the boiled water for twenty minutes to produce a tea-like drink. 0.1-300 grams (preferably 10-50 grams) of the wet hemp blend was added to water, strained and squeezed to produce a liquid. The liquid and the tea-like drink were then mixed to produce a beverage base.

### Example 26. Preparation of a Formulation of a Beverage Containing Water-Soluble Hemp and Orange Pulp Powder

Water-soluble hemp powder was prepared as described in Example 4. Water-infusing orange pulp powder was prepared as described in Example 2.

To prepare 12 oz beverage containing water-soluble hemp and orange pulp powder, 0.1-50 grams (preferably 2-10 grams) orange pulp powder was added to water and cold brewed for 12-24 hours in a fridge or stored under similar conditions to create a tea-like drink. 0.1-300 grams (preferably 10-50 grams) water-soluble hemp powder was added to water, then mixed with the tea-like drink to create a beverage base.

Another method to prepare 12 oz beverage containing water-soluble hemp and orange pulp powder is adding 0.1-50 grams (preferably 2-10 grams) coffee fruit powder and 0.1-50 grams (preferably 2-10 grams) orange pulp powder to water and bring to boil. The coffee fruit powder and orange pulp powder were then steeped in the boiled water for twenty minutes to produce a tea-like drink. 0.1-300 grams (preferably 10-50 grams) water-soluble hemp powder was added to water, then mixed with the tea-like drink to create a beverage base.

### Example 27. Preparation of a Formulation of a Beverage Containing Water-Soluble Hemp, Coffee Fruit, and Orange Pulp

Water-soluble hemp powder was prepared as described in Example 4. Water-infusing coffee fruit powder was prepared as described in Example 1. Water-infusing orange pulp powder was prepared as described in Example 2.

To prepare 12 oz beverage containing water-soluble hemp, coffee fruit and orange pulp, 0.1-50 grams (preferably 2-10 grams) orange pulp powder and 0.1-50 grams (preferably 2-10 grams) coffee fruit powder were added to water and mixed with 0.1-300 grams (preferably 10-50 grams) water-soluble hemp powder. The mixture was then cold brewed for 12-24 hours in a fridge or stored under similar conditions to create a beverage base.

Another method to prepare 12 oz beverage containing water-soluble hemp, coffee fruit and orange pulp is adding 0.1-50 grams (preferably 2-10 grams) coffee fruit powder and 0.1-50 grams (preferably 2-10 grams) orange pulp powder to water and bring to boil. The coffee fruit powder and orange pulp powder were then steeped in the boiled water for twenty minutes to produce a tea-like drink. 0.1-300 grams (preferably 10-50 grams) water-soluble hemp powder was added to distilled water, then mixed with the tea-like drink to create a beverage base.

### Example 28. Combination of Hemp-Containing Beverage with Sweeteners or Other Beverages

Hemp-containing beverages as described in this application can be combined with sweeteners such as maple syrup, cane sugar, honey or other. Hemp-containing beverages as described in this application can also be combined with other beverages, such as iced coffee latte, fruit juice and raw cacao latte or mocha latte. Milk substitutes such as coconut milk can be used. When adding the hemp-containing beverages to coffee or cacao lattes, subtle hints of coffee fruit, hemp and orange pulp can be detected, but are subtle backdrop of a predominant coffee/cacao/mocha taste.

### NUMBERED EMBODIMENTS OF THE DISCLOSURE

Notwithstanding the appended claims, the disclosure sets forth the following numbered embodiments:

1. A water infusing powder comprising:
dehydrated coffee fruit pulp powder; and
dehydrated hemp pulp powder.

2. The water infusing powder of embodiment 1, wherein the dehydrated coffee fruit pulp powder and the dehydrated hemp pulp powder were dehydrated using an ultrasound pulverization system.

3. The water infusing powder of embodiment 1, wherein the dehydrated hemp pulp powder comprises dehydrated pulp from a hemp inflorescence.

4. The water infusing powder of embodiment 1, wherein the dehydrated hemp pulp powder comprises dehydrated pulp from a hemp sun leaf

5. The water infusing powder of embodiment 1, wherein the dehydrated coffee fruit pulp powder is substantially free of dehydrated coffee bean powder.

6. The water infusing powder of embodiment 1, wherein the dehydrated coffee fruit pulp powder comprises dehydrated coffee bean powder.

7. A method for producing a water infusing powder, said method comprising:
a) providing hemp pulp, coffee fruit pulp, and ascorbic acid in a container to produce a mixture;
b) dehydrating the mixture to create a water infusing dried powder.

8. A water-soluble powder comprising:
a) dehydrated coffee fruit juice;
b) dehydrated hemp juice.

9. The water-soluble powder of embodiment 8, wherein the dehydrated coffee fruit juice had a sugar content between 2° Bx to 8° Bx before being dehydrated.

10. The water-soluble powder of embodiment 8, wherein the dehydrated coffee fruit juice had a sugar content between 3° Bx to 4° Bx before being dehydrated.

11. The water-soluble powder of embodiment 8, wherein the dehydrated hemp juice comprises dehydrated juice from a hemp inflorescences.

12. The water-soluble powder of embodiment 8, wherein the dehydrated hemp juice comprises dehydrated juice from hemp sun leaves.

13. The water-soluble powder of embodiment 8, wherein the dehydrated coffee fruit juice comprises dehydrated juice from whole coffee fruits.

14. The water-soluble powder of embodiment 8, wherein the dehydrated coffee fruit juice comprises dehydrated juice from debeaned coffee fruits.

15. A method for producing a water-soluble powder, said method comprising:
a) drying coffee fruit juice to produce dried coffee fruit powder;
b) drying hemp juice to produce dried hemp juice powder; and
c) combining the hemp juice powder with the dried coffee fruit powder to form a water-soluble dried powder.

16. A method for producing a water-soluble powder, said method comprising:
a) a) drying premixed coffee fruit juice and hemp juice to form a water-soluble dried powder.

17. The method of embodiments 15 or 16, wherein the coffee fruit juice is fortified with coffee fruit extract.

18. A hemp and coffee fruit beverage comprising:
a) coffee fruit juice; and
b) hemp juice.

19. The hemp and coffee fruit beverage of embodiment 18 further comprising sugar.

20. The hemp and coffee fruit beverage of embodiment 18, wherein the coffee fruit juice and hemp juice are mixed in a ratio of 2:1 w/w to 4:1 w/w.

21. The hemp and coffee fruit beverage of embodiment 18, wherein the coffee fruit juice and hemp juice are mixed in a ratio of 3:1 w/w.

22. The hemp and coffee fruit beverage of embodiment 18, wherein the hemp juice comprises juice from hemp inflorescences.

23. The hemp and coffee fruit beverage of embodiment 18, wherein the hemp juice comprises juice from hemp sun leaves.

24. A beverage, prepared by a method comprising contacting the water infusing powder of embodiment 1 with water.

25. A beverage, prepared by the method comprising contacting the water-soluble dried powder of embodiment 8 with water.

26. The beverage of any one of embodiments 18, and 24-25 comprising lemon juice and ginger.

27. A water-permeable container comprising the water infusing powder of any one of embodiments 1-7.

28. A gel capsule comprising the water infusing powder of any one of embodiments 1-7.

29. A gel capsule comprising the water-soluble powder of any one of embodiments 8-14.

30. A tablet comprising the water infusing powder of any one of embodiments 1-7.

31. A tablet comprising the water-soluble powder of any one of embodiments 8-14.

32. A water infusing powder comprising:
a) dehydrated coffee fruit pulp powder;
b) dehydrated hemp pulp powder; and
c) dehydrated orange pulp powder.

33. The water infusing powder of embodiment 32, wherein the dehydrated coffee fruit pulp powder, the dehydrated hemp pulp powder, and the dehydrated orange pulp powder were dehydrated using an ultrasound pulverization system.

34. The water infusing powder of embodiment 32, wherein the dehydrated hemp pulp powder comprises dehydrated pulp from a hemp inflorescence.

35. The water infusing powder of embodiment 32, wherein the dehydrated hemp pulp powder comprises dehydrated pulp from a hemp sun leaf.

36. The water infusing powder of embodiment 32, wherein the dehydrated coffee fruit pulp powder is substantially free of dehydrated coffee bean powder.

37. The water infusing powder of embodiment 32, wherein the dehydrated coffee fruit pulp powder comprises dehydrated coffee bean powder.

38. The water infusing powder of embodiment 32, wherein the dehydrated orange pulp powder comprises a whole orange.

39. The water infusing powder of embodiment 32, wherein the dehydrated orange pulp powder comprises a peeled orange.

40. The water infusing powder of embodiment 32, wherein the dehydrated orange pulp powder comprises a juiced orange.

41. The water infusing powder of embodiment 32, wherein the dehydrated orange pulp powder comprises a juiced orange without the peel.

42. A method for producing a water infusing powder, said method comprising:
a) providing hemp pulp, coffee fruit pulp, and orange pulp in a container to produce a mixture;
b) dehydrating the mixture to create a water infusing dried powder.

42a. The method of claim 42, wherein distilled water is added to the container.

43. The method of embodiment 42, wherein step a) further comprises ascorbic acid in the mixture.

44. A water-soluble powder comprising:
a) dehydrated coffee fruit juice;
b) dehydrated hemp juice; and
c) dehydrated orange juice powder.

45. The water-soluble powder of embodiment 44, wherein the dehydrated coffee fruit juice had a sugar content between 2° Bx to 8° Bx before being dehydrated.

46. The water-soluble powder of embodiment 44, wherein the dehydrated coffee fruit juice had a sugar content between 3° Bx to 4° Bx before being dehydrated.

47. The water-soluble powder of embodiment 44, wherein the dehydrated hemp juice comprises dehydrated juice from a hemp inflorescences.

48. The water-soluble powder of embodiment 44, wherein the dehydrated hemp juice comprises dehydrated juice from hemp sun leaves.

49. The water-soluble powder of embodiment 44, wherein the dehydrated coffee fruit juice comprises dehydrated juice from whole coffee fruits.

50. The water-soluble powder of embodiment 44, wherein the dehydrated coffee fruit juice comprises dehydrated juice from debeaned coffee fruits.

51. The water-soluble powder of embodiment 44, wherein the dehydrated orange juice powder comprises dehydrated juice from a whole orange.

52. The water-soluble powder of embodiment 44, wherein the dehydrated orange pulp powder comprises dehydrated juice from a peeled orange.

53. A method for producing a water-soluble powder, said method comprising:
a) drying coffee fruit juice to produce dried coffee fruit powder;
b) drying hemp juice to produce dried hemp juice powder;
c) drying orange juice to produce a dried orange juice powder; and
d) combining the hemp juice powder, dried coffee fruit powder, and dried orange juice powder to form a water-soluble dried powder.

54. A method for producing a water-soluble powder, said method comprising:
a) a) drying premixed coffee fruit juice, hemp juice, and orange juice to form a water-soluble dried powder.

55. The method of embodiments 53 or 54, wherein the coffee fruit juice is fortified with coffee fruit extract.

56. A hemp, coffee fruit, and orange beverage comprising:
a) coffee fruit juice;
b) hemp juice; and
c) orange juice.

57. The hemp, coffee fruit, and orange beverage of embodiment 56 further comprising sugar.

58. The hemp, coffee fruit, and orange beverage of embodiment 56, wherein the coffee fruit juice and hemp juice are mixed in a ratio of 2:1 w/w to 4:1 w/w.

59. The hemp, coffee fruit, and orange beverage of embodiment 56, wherein the coffee fruit juice and hemp juice are mixed in a ratio of 3:1 w/w.

60. The hemp, coffee fruit, and orange beverage of embodiment 56, wherein the hemp juice comprises juice from hemp inflorescences.

61. The hemp, coffee fruit, and orange beverage of embodiment 56, wherein the hemp juice comprises juice from hemp sun leaves.

62. The hemp, coffee fruit, and orange beverage of embodiment 56, wherein the orange juice comprises juice from a whole orange.

63. The hemp, coffee fruit, and orange beverage of embodiment 56, wherein the dehydrated orange juices comprise juices from a peeled orange.

64. A beverage, prepared by a method comprising contacting the water infusing powder of any one of embodiments 32-41 with water.

65. A beverage, prepared by the method comprising contacting the water-soluble dried powder of any one of embodiments 44-52 with water.

66. The beverage of any one of embodiments 56-65 comprising lemon juice and ginger.

67. A water-permeable container comprising the water infusing powder of any one of embodiments 32-41.

68. A gel capsule comprising the water infusing powder of any one of embodiments 32-41.

69. A gel capsule comprising the water-soluble powder of any one of embodiments 44-52.

70. A tablet comprising the water infusing powder of any one of embodiments 32-41.

71. A tablet comprising the water-soluble powder of any one of embodiments 44-52.

72. A method of producing dehydrated orange pulp powder, said method comprising the steps of:
a) providing pre juiced orange tissue, and
b) exposing said tissue to ultrasonic waves,
wherein the ultrasonic waves simultaneously pulverize and dehydrate the orange tissue, thereby producing dehydrated orange pulp powder.

73. The method of embodiment 72, wherein the ultrasonic waves are at a frequency from about 1 kHz to about 20 kHz.

74. The method of embodiment 72 or 73, wherein the ultrasonic waves are emitted from an ultrapulverization machine.

75. The method of any one of embodiments 72-74, further comprising the step of heating the dehydrated pulp powder in a microwave dehydrator.

What is claimed is:

1. A composition comprising a hemp content, wherein
the hemp content is selected from
an insoluble hemp pulp powder made from a hemp inflorescence or a hemp sun leaf,
a hemp juice made from a hemp inflorescence or a hemp sun leaf,
a soluble hemp juice powder made from a hemp inflorescence or a hemp sun leaf, and
a wet hemp blend containing water, pulverized hemp stems and pulverized hemp leaves.

2. A composition according to claim 1, further comprising a coffee content, wherein
the coffee content is selected from
an insoluble coffee fruit pulp powder made from whole coffee fruits or debeaned coffee fruits,
a coffee fruit juice made from whole coffee fruits or debeaned coffee fruits, and
a soluble coffee fruit powder made from whole coffee fruits or debeaned coffee fruits.

3. The composition according to claim 2, wherein the composition is in the form of
a powder,
a gel capsule,
a tablet, or
a water-permeable container.

4. The composition according to claim 2, further comprising at least one ingredient selected from
ascorbic acid;
lemon juice and ginger;
coffee fruit extract; and
sweeteners such as sugar, maple syrup, cane sugar, honey.

5. The composition according to claim 1, further comprises an orange content, wherein the orange content is selected from:
an insoluble orange pulp powder made from whole oranges, peeled oranges, juiced oranges or juiced oranges without the peel;
an orange juice made from whole oranges or peeled oranges; and
a soluble orange juice powder made from whole oranges or peeled oranges.

6. The composition according to claim 2, wherein the coffee content has a sugar content between 2° Bx to 8° Bx before being dehydrated.

7. The composition according to claim 2, wherein the coffee content has a sugar content between 3° Bx to 4° Bx before being dehydrated.

8. The composition according to claim 2, wherein
the coffee content is a coffee fruit juice,
the hemp content is a hemp juice,
the coffee fruit juice and hemp juice are mixed in a ratio of 2:1 w/w to 4:1 w/w.

9. The composition according to claim 2, wherein
the coffee content is a coffee fruit juice,
the hemp content is a hemp juice,
the coffee fruit juice and hemp juice are mixed in a ratio of 3:1 w/w.

10. The composition according to claim 2, wherein the coffee content and the hemp content were dehydrated using an ultrasound pulverization system.

11. The composition according to claim 1, further comprising a dehydrated orange content, wherein the dehydrated orange content was dehydrated using an ultrasound pulverization system.

12. The composition according to claim 2, wherein
the concentration of the hemp content is between 0.1-300 g per 12 oz of the composition;
the concentration of the coffee content is between 0.1-50 g per 12 oz of the composition.

13. The composition according to claim 2, wherein
the concentration of the hemp content is between 10-50 g per 12 oz of the composition;
the concentration of the coffee content is between 2-10 g per 12 oz of the composition.

14. The composition according to claim 5, wherein
the concentration of the hemp content is between 0.1-300 g per 12 oz of the composition;
the concentration of the orange content is between 0.1-50 g per 12 oz of the composition.

15. The composition according to claim 5, wherein
the concentration of the hemp content is between 10-50 g per 12 oz of the composition;
the concentration of the orange content is between 2-10 g per 12 oz of the composition.

16. A method for making a composition, said method comprises the simultaneous and/or sequential steps of:
a) providing a hemp content selected from
an insoluble hemp pulp powder made from a hemp inflorescence or a hemp sun leaf,
a hemp juice made from a hemp inflorescence or a hemp sun leaf,
a soluble hemp juice powder made from a hemp inflorescence or a hemp sun leaf, and
a wet hemp blend containing water, pulverized hemp stems and pulverized hemp leaves;
b) providing at least one of a coffee content or an orange content to create a mixture with the hemp content,
wherein the coffee content is selected from
an insoluble coffee fruit pulp powder made from whole coffee fruits or debeaned coffee fruits,
a coffee fruit juice made from whole coffee fruits or debeaned coffee fruits, or
a soluble coffee fruit powder made from whole coffee fruits or debeaned coffee fruits;
wherein the orange content is selected from
an insoluble orange pulp powder made from whole oranges, peeled oranges, juiced oranges or juiced oranges without the peel,
an orange juice made from whole oranges or peeled oranges,
a soluble orange juice powder made from whole oranges or peeled oranges.

17. The method of claim 16, further comprising at least one of the steps:
a) dehydrating the mixture using an ultrasound pulverization system;
b) heating the mixture in a microwave dehydrator.

18. The method of claim 16, further comprising the steps:
a) add water to the mixture and cold brew for 1-48 hours,
b) strain and squeeze the mixture.

19. The method according to claim 16, further comprising: contacting the mixture with water to create a beverage.

20. A method of inducing health benefits in a human, said method comprising:
a) providing the composition made with the method according to claim 16; and
b) administering the composition to the human;
wherein the health benefits include relieving chronic pain, improving lung capacity, stimulating weight loss, regulating and/or preventing diabetes, stabilizing moods, treating autism, treating depression, regulating seizures, healing broken bones, improving concentration and/or focus, treating glaucoma, alleviating anxiety, slowing development of Alzheimer's disease, alleviating the pain associated with arthritis and/or multiple sclerosis, treating posttraumatic stress disorder symptoms, treating inflammatory bowel diseases, combating alcoholism, and reducing the tremors associated with Parkinson's Disease.
